(19) United States
(12) Patent Application Publication
(10) Pub. No.: US 2025/0009771 A1
(43) Pub. Date: Jan. 9, 2025

(54) PHARMACEUTICAL COMPOSITION FOR TREATING COLORECTAL CANCER, INCLUDING TUMOR-TARGETED LIPOSOMES CONTAINING PLATYCODIN D2

(71) Applicants: INDUSTRY-UNIVERSITY COOPERATION FOUNDATION HANYANG UNIVERSITY ERICA CAMPUS, Ansan-si (KR); RESEARCH & BUSINESS FOUNDATION SUNGKYUNKWAN UNIVERSITY, Suwon-si (KR)

(72) Inventors: Chul-Su YANG, Ansan-si (KR); Jin Woong Kim, Seongnam-si (KR); Seok-Jun Mun, Ansan-si (KR); Hyo Keun Kim, Ansan-si (KR); Euni Cho, Ansan-si (KR); Minha Jeon, Suwon-si (KR)

(73) Assignees: INDUSTRY-UNIVERSITY COOPERATION FOUNDATION HANYANG UNIVERSITY ERICA CAMPUS, Ansan-si (KR); RESEARCH & BUSINESS FOUNDATION SUNGKYUNKWAN UNIVERSITY, Suwon-si (KR)

(21) Appl. No.: 18/665,849

(22) Filed: May 16, 2024

(30) Foreign Application Priority Data
Apr. 10, 2023 (KR) 10-2024-005367
May 17, 2023 (KR) 10-2023-0063601

(51) Int. Cl. A61K 31/704; A61K 9/127; A61K 47/69; A61P 35/00

(52) U.S. Cl. CPC A61K 31/704; A61K 9/127; A61K 47/6911; A61P 35/00

(57) ABSTRACT

The present invention relates to a pharmaceutical composition for treating cancer, including platycodin D2 as a pharmaceutically active ingredient, and liposomes containing platycodin D2. It was confirmed that the composition or liposomes according to the present invention may be conjugated with an acidity-triggered rational membrane (ATRAM) to deliver a drug in a tumor cell-specific manner and allow the drug to function, and is particularly effective in treating colorectal cancer.

### CROSS-REFERENCE TO RELATED APPLICATION

This application claims priority to and the benefit of Korean Patent Application No. 2023-0063601, filed on May 17, 2023, and Korean Patent Application No. 2024-0053657, filed on Apr. 22, 2024, the disclosure of which is incorporated herein by reference in its entirety.

### INCORPORATION BY REFERENCE OF SEQUENCE LISTING

The content of the electronically submitted sequence listing, file name: 020240186US-DP240063.xml; size: 8.9 KB; and date of creation: Aug. 2, 2024, filed herewith, is incorporated herein by reference in its entirety.

### BACKGROUND

### 1. Field of the Invention

The present invention relates to a pharmaceutical composition for treating colorectal cancer, including tumor-targeted liposomes containing platycodin D2.

### 2. Discussion of Related Art

Colorectal cancer (CRC) is a leading cause of cancer-related mortality. The balance between the rates of cell growth and apoptosis that maintains intestinal epithelial cell homeostasis is an important factor in the development of CRC, which means that dysregulated apoptosis is an important factor in CRC. B-cell lymphoma-2 (BCL-2) family proteins, known to be the key regulators of intrinsic apoptosis, are overexpressed in CRC lesions, thereby hindering the efficacy of various chemotherapeutics. Therefore, BCL-2 is an important target for CRC therapy. Currently, the target of chemotherapeutics for CRC treatment is the apoptosis pathway, however, their efficacy is frequently hindered by unfavorable pharmacokinetics and limited targeting efficiency, which results in increased incidence of toxic side effects due to frequent drug administration. Novel approaches are required to overcome dysregulated apoptosis signaling and increase the efficacy of CRC treatment.

Phytochemicals are naturally occurring compounds obtained from plants, and plant saponins, a type of phytochemical, are known to be vital resources in the development of anticancer agents due to their characteristics such as biological activity, minimal side effects, and low cost. Platycosides (PS) are triterpenoid saponins extracted from balloon flower roots, and are known to exhibit significant anticancer potency in various types of cancers, including breast cancer, lung cancer, and colorectal cancer. These bioactive platycosides have been proven to promote tumor cell death by regulating various apoptosis-related factors, serving as potent apoptosis-inducing anticancer agents. However, due to poor permeability and low stability, the application of PS as an antitumor agent is considerably limited.

Fortunately, nanocarriers (drug delivery systems), especially liposomes, can serve as platforms for delivery of these phytochemicals. Increasing evidence supports the fact that encapsulation of saponins with liposomes improve their solubility, bioavailability and enhance therapeutic efficacy. Currently, most anticancer nanotherapeutics rely on “passive” targeting strategies, which rely solely on the enhanced permeability and retention (EPR) effect to reach the tumor site. However, this strategy can be severely limited by tumor complexity and heterogeneity. Only a small percentage of these nanotherapeutics have been observed to be able to penetrate high-EPR xenografted tumors (less than 1% according to a recent meta-analysis study), necessitating active tumor-targeting strategies. One such active targeting strategy involves enhancing the surface functionalization of liposomes by using tumor-targeting ligands, especially peptides, which can help overcome the current limitations of nanocarriers in the treatment of solid tumors. The pH-responsive acidity-triggered rational membrane (ATRAM) peptide has been proven to successfully deliver liposomes via pH-responsive membrane interactions into cancer cells within the acidic tumor microenvironment (TME). The present inventors have previously demonstrated that an ATRAM-conjugated multifunctional GRA8 peptide successfully targets tumor cells in vitro and in vivo.

### RELATED ART DOCUMENTS

### Non-Patent Document

Vanessa P. Nguyen, et al., Biochemistry 2015, 54, 6567-6575

### SUMMARY OF THE INVENTION

The present invention is directed to providing an anticancer agent or pharmaceutical composition for cancer treatment, specifically acting on colorectal cancer.

The present invention is directed to a pharmaceutical composition for treating cancer, including platycodin D2 as a pharmaceutically active ingredient, wherein the cancer is colorectal cancer, breast cancer, or lung cancer; in another embodiment of the present invention, the cancer is colorectal cancer; in still another embodiment of the present invention, the platycodin D2 is encapsulated in a liposome; in yet another embodiment of the present invention, the platycodin D2 is fixed to a lipid bilayer of the liposome; in yet another embodiment of the present invention, the liposome further includes an acidity-triggered rational membrane (ATRAM) protein; and in yet another embodiment of the present invention, the ATRAM protein includes an amino acid sequence represented by SEQ ID NO: 1.

The present invention includes a liposome including platycodin D2. In one embodiment of the present invention, the platycodin D2 is fixed to a lipid bilayer of the liposome; in another embodiment of the present invention, the liposome further includes an ATRAM protein; in still another embodiment of the present invention, the ATRAM protein includes an amino acid sequence represented by SEQ ID NO: 1; and in yet another embodiment of the present invention, the liposome may be included in a pharmaceutical composition as an active ingredient for treating cancer.

According to the present invention, an ATRAM protein-conjugated liposome containing platycodin D2, and a pharmaceutical composition for treating colorectal cancer including the same exhibit cancer-specific apoptotic ability, and thus can be used as an anticancer agent with suppressed non-specific reactions.

### BRIEF DESCRIPTION OF THE DRAWINGS

FIGS. 1A-1F. Preparation and characterization of PS-Lipo. FIG. 1A is a schematic diagram of platycoside-containing liposomes (PS-Lipo) for induction of cancer cell cytotoxicity. (B) Hydrodynamic particle size of C-Lipo, PCD-Lipo, PCD2-Lipo, and PGD-Lipo. (C) Transmission electron microscopy (TEM) images of C-Lipo, PCD-Lipo, PCD2-Lipo, and PGD-Lipo. Scale bars are 100 nm. (D) Average hydrodynamic particle size of C-Lipo, PCD-Lipo, PCD2-Lipo, and PGD-Lipo on day 1 and day 15. Mean±SD is shown, and significance was determined by a paired t-test (C-Lipo vs. PCD-Lipo, PCD2-Lipo, and PGD-Lipo). *p<0.05. (E) Quantitative analysis of cellular uptake of Texas Red-loaded C-Lipo, PCD-Lipo, PCD2-Lipo, or PGD-Lipo by macrophages (BMDM and Raw264.7) and cancer cells (DLD-1 and CT26) at 5 μM. (F) Representative confocal laser scanning microscope (CLSM) images of cellular uptake of Texas Red-loaded C-Lipo, PCD-Lipo, PCD2-Lipo, or PGD-Lipo by cancer cells (DLD-1 and CT26). Scale bars, 75 μm.

FIGS. 2A-2E. In vitro anticancer activity of platycoside liposomes. In vitro cytotoxicity of free PS and PS-Lipo against macrophages (FIGS. 2A-2B) and CRC cells (FIGS. 2C-2D) was evaluated by WST-8 cell viability and lactate dehydrogenase (LDH) assay after 48 h of co-culture. Inset: IC₅₀ values (μM) are shown. (E) Western blot (WB) analysis (left) of MCL-1, BCL-2, and BCL-X_{L} expression in DLD-1 and CT26 cells after treatment of 10 μM and 5 μM PCD2-Lipo for 10 h and 8 h and quantification of WB protein levels (right).

FIG. 3. Cytotoxicity of PS-Lipo against various cancer cells. In vitro cytotoxicity of PS-Lipo against A549 and MB231 was evaluated by WST-8 viability assay during 48 h culture. Inset: IC₅₀ values (μM) are shown.

FIGS. 4A-4I. Inhibition of intrinsic apoptosis (caspase-9) abolished PS-Lipo-induced apoptosis. (A) Schematic representation of the apoptotic pathway. Apoptosis can be induced by either an extrinsic pathway through death receptors that specifically activate caspase-8 or an intrinsic pathway through expression of BCL2 family BH3-only proteins (BCL-2, BCL-XL, and MCL-1) that leads to activation of caspase-9. (FIGS. 4B & 4D) WB analysis of caspase-8, caspase-9, caspase-3, PARP, and GAPDH protein expression in DLD-1 cells (B) or CT26 (D) after different PS-Lipo treatments for 8 h. (FIGS. 4C & 4E) Quantification of WB protein levels by group in DLD-1 (C) and CT26 (E) cells. (F & H) Cellular cytotoxicity of PCD2-Lipo against wild-type (WT) or caspase-9 knockout (KO) DLD-1 cells (F) and CT26 cells was evaluated by WST-8 viability assay after 48 h of co-culture (H). (FIGS. 4G & 4I) WB analysis of caspase-8, caspase-9, caspase-3, PARP, and GAPDH protein expression in WT or caspase-9 KO DLD-1 (G) or CT26 cells (I) after different PCD-Lipo treatments for different times.

FIGS. 5A-5D. Caspase-9-deficient CRC cell lines generated using the CRISPR-Cas9 system were used for in vitro cytotoxicity assays and establishment of tumor xenograft models. (FIGS. 5A & 5B) Quantification of WB protein levels or mRNA expression levels of caspase-9 by the generated caspase-9-deficient DLD-1 (C) and CT26 (D); GAPDH denotes a loading control. Mean±SD is shown, and significance was determined by an unpaired t-test (WT vs. caspase-9 KO). **p<0.05, ***p<0.001, and ****p<0.0001. (FIGS. 5C & 5D) Growth curves of DLD-1 or CT26 parents (WT) or caspase-9 KO clones. 1×10⁴ cells were cultured in 100 mm³ plates with 10% FBS for the indicated number of days. The number of cells was counted every 24 hours using a hemocytometer. Mean±SD is shown, and significance was determined by a paired t-test (WT vs. caspase-9 KO).

FIGS. 6A-6K. Tumor-targeting PCD2-Lipo-ATRAM inhibits xenograft CRC tumor growth. (A) Schematic illustration of PCD2-Lipo-ATRAM for targeting an acidic tumor microenvironment by pH-responsible membrane interaction. Preparation and characterizations of tumor-targeting PCD2-Lipo-ATRAM. (B) Characterization of PCD2-Lipo-ATRAM. (C) Schematic diagram of biodistribution imaging in DLD-1 xenograft model. (D) In vivo near-infrared (NIR) fluorescent images of nude mice at 1, 12, 24, and 48 h after the administration of PBS, PCD2-Lipo, and PCD2-Lipo-ATRAM; n=3 or 4 per group. (E) Representative ex vivo NIR fluorescent images of organs (heart, lung, liver, spleen, and kidney) and excised tumors. (F) In vivo & ex vivo average radiant efficiency of NIR fluorescent images. In vivo NIR fluorescent average radiant efficiency of tumor (24 h after administration). Mean±SD is shown, and significance was determined by an unpaired t-test (PCD2-Lipo vs. PCD2-Lipo-ATRAM). ***p<0.001. Ex vivo NIR fluorescent average radiant efficiency of tissue (heart, lung, liver, spleen, kidney and tumor 24 h after administration). Mean±SD is shown, and significance was determined by an unpaired t-test (Tumor PCD2-Lipo vs. Tumor PCD2-Lipo-ATRAM). **p<0.05. (G) Schematic diagram of tumor therapy regimen in CRC xenografts (DLD-1 or CT26). PBS (control), PCD2-Lipo, or PCD2-Lipo-ATRAM was intraperitoneally administered to wild-type (WT) or caspase-9 knockout (KO) nude mice after tumor establishment (50 to 150 mm³). (H) Tumor progression was closely monitored by weekly tumor volume measurement using calipers under a 28-day treatment regimen (n=5 to 10 per group). Mean±SEM is shown, and significance was determined by two-way analysis of variance (ANOVA) (WT+PCD2-Lipo-ATRAM vs. WT+PCD2-Lipo and WT+PCD2-Lipo-ATRAM vs. Casp9 KO+PCD2-Lipo-ATRAM). **** denotes p<0.0001. (I) Images of excised xenografted CRC tumors in the five groups at the end point (day 28). (J) WB analysis of caspase-9, caspase-8, caspase-3, PARP, and GAPDH protein expression in the five groups. (K) Immunohistochemical staining of tumor sections for hematoxylin and eosin (H&E), cell proliferation markers (Ki-67 and PCNA), and apoptosis markers (cleaved caspase-3 and cleaved PARP). Images were taken at 20×magnification. Scale bar, 50 μm.

### DETAILED DESCRIPTION OF EXEMPLARY EMBODIMENTS

Hereinafter, the present invention will be described in detail through embodiments of the present invention with reference to the attached drawings. However, the following embodiments are provided as examples of the present invention, and when it is judged that a detailed description of a technology or configuration well known to those skilled in the art may unnecessarily obscure the gist of the present invention, the detailed description may be omitted, and the present invention is not limited thereby. Various modifications and applications of the present invention are possible within the scope of the claims described below and the equivalents interpreted therefrom.

In addition, the terms used in the present specification are terms used to appropriately express preferred embodiments of the present invention, and may vary depending on the intention of the user or operator or the practices of the field to which the present invention pertains. Therefore, definitions of these terms should be made based on the content throughout the present specification. Throughout the present specification, when a part is said to “include” a certain component, unless specifically stated to the contrary, this means that it may further include other components rather than excluding other components.

All technical terms used in the present invention, unless otherwise defined, are used with the same meaning as commonly understood by one of ordinary skill in the art in the field related to the present invention. In addition, although preferred methods and samples are described in the present specification, similar or equivalent methods and samples are also included in the scope of the present invention. The contents of all publications incorporated in the present specification by reference are incorporated in the present invention.

In the present invention, platycoside (PS)-based liposomes were designed based on the versatile properties of PS and its applicability in colorectal cancer CRC therapy. Three PS types, platycodin D (PCD), platycodin D2 (PCD2), and polygalacin D (PGD), which are commonly used in cancer therapy, were optimized in the present invention. The present inventors hypothesized that the encapsulation of PS with liposomes (PS-Lipo) could help overcome their disadvantages, such as poor stability and limited permeability, by facilitating intracellular uptake, thereby enhancing their inherent antitumor efficacy. The in vitro mechanistic studies by the present inventors revealed that PS-Lipo induces apoptotic tumor cell death through the caspase-9/3 axis, demonstrating that the mechanism of PS-Lipo relies on the intrinsic apoptotic pathway. Since the platycodin D2-based liposome (PCD2-Lipo) exhibited the strongest antitumor efficacy compared to other PS-Lipo molecules, an acidity-triggered rational membrane (ATRAM)-conjugated PCD2-Lipo (PCD2-Lipo-ATRAM) was constructed for tumor-targeting therapy. In CRC xenografts (DLD-1 & CT26), PCD2-Lipo-ATRAM successfully inhibited CRC tumor growth by targeting the tumor sites. The pH-responsive PCD2-Lipo-ATRAM may be used as a promising cancer drug delivery platform that combines stability with effective tumor-targeting and induces the apoptosis of cancer cells.

The present invention relates to a pharmaceutical composition including platycodin D2 as a pharmaceutical active ingredient and using a liposome conjugated with an ATRAM peptide as a carrier capable of specifically delivering the same to cancer tissue. ATRAM may be conjugated with the liposome using a mannosyl erythritol lipid (MEL)-maleimide linker, but is not limited thereto, and may be synthesized and conjugated according to methods known in the art. In addition, platycodin D2 is present in a liposome, and in particular, in the present invention, it is loaded in a way that penetrates the membrane of the liposome. However, as long as platycodin D2 can have the same level of pharmacological activity, it may be used by being conjugated with the surface of the membrane or modified to be positioned within the membrane.

In the present invention, platycodin D, or PCD, refers to a compound having a structure represented by the following Chemical Formula 1.

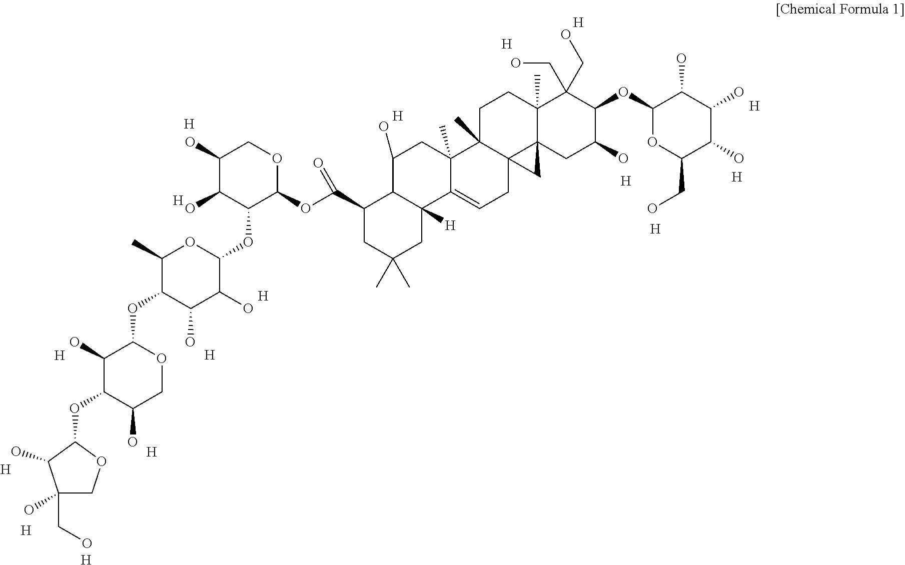

In the present invention, platycodin D2, or PCD2, refers to a compound having a structure represented by the following Chemical Formula 2.

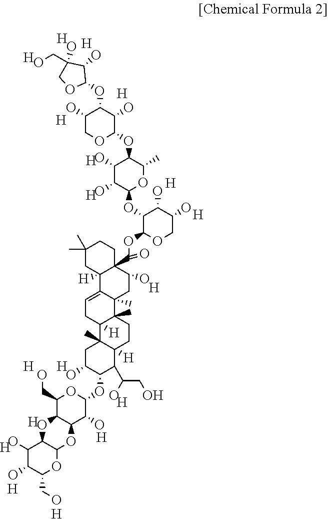

In the present invention, polygalacin D, or PGD, refers to a compound having a structure represented by the following Chemical Formula 3.

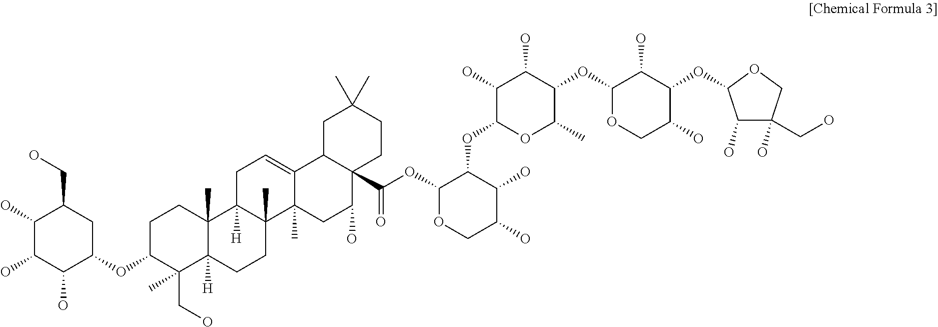

It is desirable to design a tumor-targeting delivery system that not only selectively targets an acidic tumor microenvironment (TME), but also promotes tumor apoptosis, ultimately destroying tumor cells while sparing normal cells. Recently, safe, biodegradable, and biocompatible drug delivery systems for targeted and effective antitumor therapies have been successfully created by combining bioactive natural platycosides and nanoscience. In the present invention, a new tumor-targeted liposomal delivery system including platycoside PCD2 as an active ingredient for CRC therapy was developed. To achieve tumor-targeting ability, the shell of liposomes including PCD2 was functionalized with an ATRAM peptide to promote internalization into cancer cells within the acidic TME. The liposomes including PCD2 and conjugated with ATRAM successfully reached the tumor site by targeting the acidic TME and also exhibited potent apoptotic activity against CRC cells.

The ATRAM peptide includes or consists of an amino acid sequence represented by SEQ ID NO: 1.

SEQ ID NO: 1:
N′-GLAGLAGLLGLEGLLGLPLGLLEGLWLGLELEGN-C′

ATRAM peptides have high solubility in solutions and are capable of interacting with lipid membranes in a pH-dependent manner. ATRAM peptides are conjugated with the surface of lipid membranes at pH 8.0, but when acidified, they are inserted into the lipid bilayer and change into a transmembrane α-helix structure. The insertion of ATRAM into the cell membrane occurs at slightly acidic pH (pK 6.5), a condition similar to the extracellular pH of solid tumors. Information about ATRAM may be found in V. P. Nguyen, et al., Biochemistry 2015, 54, 6567-6575; and V. P. Nguyen, et al., J Control Release 298 (2019) 142-153.

An embodiment of the present invention shows that anticancer phytochemicals benefit from reduced toxicity and improved tumor uptake when encapsulated in liposomes. Poor physicochemical properties and stability along with poor solubility have limited the use of PCD2 as a potent therapeutic agent against CRC. The present inventors confirmed that PCD2 itself exhibited cytotoxicity toward both CRC and normal cells.

However, when PCD2 was encapsulated in a liposome, the cytotoxicity and proapoptotic efficacy of PCD2 on tumor cells was greatly enhanced in vitro, but did not affect normal cells. PCD2 encapsulated in a liposome showed more potent cytotoxicity against CRC cells than other cancer types in vitro. Based on Western blot (WB) analysis results, it was observed that the potent cytotoxic effect of PCD2 on CRC could be attributed to the inhibition of antiapoptotic BCL-2 family proteins. Resistance to apoptosis by the upregulation of BCL-2 family proteins has been commonly observed in several tumor types. Recently, several studies have reported the upregulation of BCL-XL in colorectal lesions. It has also been observed that targeting BCL-XL with the highly specific BCL-XL antagonist A-1155463 significantly impaired the clonogenic potential of adenomas and tumor organoids. These results suggest that CRC tumors seem to rely on BCL-XL overexpression as a strategy for evading apoptosis. The inhibition of BCL-2 family proteins, particularly BCL-XL, by PCD2 causes the apoptosis of CRC cells, which is beneficial for CRC therapy. Therefore, PCD2 has potential as a CRC therapeutic agent.

Drug resistance is a common issue in anticancer therapy. Because of the molecular complexities of the tumor microenvironment, single-drug chemotherapy often triggers and reinforces alternative molecular pathways in cancer cells, resulting in drug-resistant mutations. Recently, combination drug delivery systems which can target multiple pathways of cancer using multiple drugs simultaneously have been extensively investigated. Liposomes have a unique ability to encapsulate hydrophilic agents in their inner aqueous core and hydrophobic drugs in their lamellae, making them versatile therapeutic carriers for combination cancer therapies. Many studies have suggested that the combination of saponins and chemotherapy could significantly enhance the chemosensitivity of tumors to clinically used anticancer drugs such as cisplatin, paclitaxel, doxorubicin, and docetaxel. It has been previously reported that the combination of platycodin D and cetuximab exhibited an obvious synergistic effect on KRAS-mutant CRC cells, potently inhibiting tumorigenesis in CRC xenograft models. In the present study, the present inventors discovered that PCD2 not only is an anticancer agent but also has potential as a membrane stabilizer. Liposomal formulations including PCD2 enhance the stability of liposomes and also benefit the delivery of anticancer drugs by increasing circulation stability and targeting efficiency. Following internalization into CRC cells, PCD2 exerted its inherent proapoptotic activity, suggesting that PCD2-based liposomes have the potential to exert their synergistic antitumor efficacy with commercial anticancer drugs by acting as a chemosensitizer of anticancer drugs to maximize their chemotherapeutic effect. Therefore, PCD2-Lipo-ATRAM may act as a biocompatible chemotherapeutic drug nanocarrier for combined cancer therapy.

Nontargeting liposomes are nanomedicine formulations approved for clinical use that rely on enhanced permeability and retention (EPR)-based accumulation in tumors, which is critically influenced by the size of nanoparticles (100 to 200 nm). The size distribution of PCD2-Lipo is 100 to 110 nm, which seems suitable for achieving the EPR effect in solid tumors. However, although PCD2-Lipo exhibited enhanced anticancer activity against CRC cells in vitro, it did not exhibit the same efficacy in vivo. When administered in vivo, it failed to reach the tumor site, which adversely affected its anticancer effect. The above results suggest that tumor accumulation based on the size-dependent EPR effect may not be sufficient for achieving effective targeting at tumor sites. Thus, to ensure effective in vivo functionality, it is crucial to not only consider the size and stability of liposomes, but also modify the targeting strategy considering the complexity of tumor biology.

One characteristic of solid tumors that may be exploited as a tumor-targeting strategy for enhancing the therapeutic benefits of nanomedicine is the acidic niche of the TME, which mainly arises from the high glycolytic rate of tumor cells. To facilitate specific tumor-targeting, PCD2-Lipo was functionalized with the pH-responsive ATRAM peptide, which may penetrate the lipid membrane under acidic conditions. After pH-dependent insertion, some of these peptides are taken up by endocytosis, particularly when the peptides are linked to liposomes. Conjugation of the pH-responsive ATRAM peptide with PCD2-Lipo maximized the tumor inhibiting effect of PCD2 by showing successful tumor-targeting and accumulation in the CRC xenografts. This was further verified in an in vivo fluorescence imaging study, where a stronger fluorescence signal of IR783-labeled liposomes was observed in the PCD2-Lipo-ATRAM group than that in PCD2-Lipo.

The present inventors developed a PCD2-based liposomal system as a targeted tumor therapy for CRC. The PCD2-Lipo-ATRAM system demonstrated successful tumor-targeting ability in CRC xenografts, which may maximize the tumor inhibition effect of PCD2 by initiating (reactivating) tumor apoptosis. PCD2 functions not only as a proapoptotic agent but also as a membrane stabilizer when combined with liposomes. PCD2-based liposomes offer a novel platform for anticancer drug delivery and may lead to a new era of nanocarrier treatment for cancer.

Liposomes are closed artificial spherical vesicles made of a lipid bilayer that mimics cell membranes and may be synthesized in sizes ranging from 50 nm to 1000 nm. For biological applications such as drug delivery, the optimal size range is 50 nm to 500 nm and may be used for packaging and delivery of various types of molecules, including small molecules, peptides, RNA, DNA, diagnostics, and therapeutics (Luk, B T., et al., Cancer Theranostics, 2012. 2(12): p. Al-Jamal, et al., Acc Chem Res, 2011. 44(10): p. 1094-104).

Liposomal nanocarriers have been developed to mitigate side effects, improve delivery, and reduce off-target toxicity based on active or passive targeting mechanisms. An example of a liposomal nanocarrier is Doxil®, the brand name for the liposome-encapsulated topoisomerase inhibitor doxorubicin, which is an FDA-approved drug used for the treatment of certain breast and pediatric cancers (Chang, H. I., et al., Int J Nanomedicine, 2012. 7: p. 49-60; Porter, C. J., et al., FEBS Letters, 1992. 305(1): p. 62-69).

In one embodiment of the present invention, the liposome includes a relevant drug (e.g., the relevant drug may be encapsulated within the liposome, incorporated into the lipid layer, or covalently attached to lipids on the surface of the liposome). In a specific embodiment, the relevant drug may be a phytochemical, more specifically a platycoside, more specifically platycodin D, platycodin D2, or polygalacin D, and in one embodiment of the present invention, platycodin D2.

In a specific embodiment, the liposome has an average diameter ranging from 30 nm to 300 nm. In a specific embodiment, the liposome has an average diameter ranging from 50 nm to 200 nm. In a specific embodiment, the liposome has an average diameter ranging from about 80 nm to 150 nm. In a specific embodiment, the liposome has an average diameter of about 90 nm. In a specific embodiment, the liposome has an average diameter of about 140 nm.

The terms “oligopeptide,” “polypeptide,” “peptide,” and “protein” used in the present invention are used interchangeably and refer to polymeric compounds consisting of amino acid residues covalently linked through peptide bonds.

In the present invention, “amino acid” refers to both L- and D-isomers of natural amino acids and other amino acids used in the peptide field to produce synthetic peptides, such as natural amino acids, non-natural amino acids, and amino acids not encoded by a base sequence.

The natural amino acids may be alanine (Ala, A), cysteine (Cys, C), aspartic acid (Asp, D), glutamic acid (Glu, E), phenylalanine (Phe, F), glycine (Gly, G), histidine (His, H), isoleucine (Ile, I), lysine (Lys, K), leucine (Leu, L), methionine (Met, M), asparagine (Asn, N), proline (Pro, P), glutamine (Gln, Q), arginine (Arg, R), serine (Ser, S), threonine (Thr, T), valine (Val, V), tryptophan (Trp, W), and tyrosine (Tyr, Y).

The other amino acids may be 2-aminoadipic acid (2-aminohexanedioic acid), α-asparagine, 2-aminobutanoic acid, 2-aminocaproic acid (2-aminodecanoic acid), α-glutamine, α-aminoisobutyric acid, α-methylalanine, 2-aminopimelic acid (2-aminohepanedioic acid), γ-amino-β-hydroxybenzenepentanoic acid, 2-aminosuberic acid (2-aminooctanedioic acid), 2-carboxyazetidine, β-alanine, β-aspartic acid, 3,6-diaminohexanoic acid, β-lysine, butanoic acid, 4-amino-3-hydroxybutanoic acid, γ-amino-β-hydroxycyclohexanepentanoic acid, 3-cyclohexylalanine, N5-aminocarbonylornithine, 3-sulfoalanine, 2,4-diaminopropanoic acid, 2,7-diaminosuberic acid (2,7-diaminooctanedioic acid), S-ethylthiocysteine, γ-glutamic acid, γ-carboxyglutamic acid, hydroxyacetic acid (glycolic acid), pyroglutamic acid, homogrginine, homocysteine, homohistidine, 2-hydroxyisovaleric acid, homoserine, 2-hydroxypentanoic acid, 5-hydroxylysine, 4-hydroxyproline, isovaline, 2-hydroxypropanoic acid (lactic acid), mercaptoacetic acid, mercaptobutanoic acid, 3-hydroxy-4-methylproline, mercaptopropanoic acid, 3-naphthylalanine, norleucine, nortyrosine, norvaline, 2-carboxyoctahydroindole, ornithine, penicillamine (3-mercaptovaline), 2-phenylglycine, 2-carboxypiperidine, sarcosine (N-methylglycine), 1-amino-1-carboxycyclopentane, statin (4-amino-3-hydroxy-6-methylheptanoic acid), 3-thienylalanine, 3-carboxyisoquinoline, 3-methylvaline, ε-N-trimethyllysine, 3-thiazolylalanine, α-amino-2,4-dioxopyrimidinepropanoic acid, and the like.

The peptides of the present invention may also be present in a salt form. Salt forms usable in the present invention may be those produced during the final isolation and purification of the compound or by reacting the amino group with an appropriate acid. For example, acid addition salts may be acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethanesulfonate, lactate, maleate, mesitylene sulfonate, methanesulfonate, naphthalenesulfonate, nicotinate, 2-naphthalenesulfonate, oxalate, pamoate, pectinate, persulfate, 3-phenylpropionate, picrate, pivalate, propionate, succinate, tartrate, trichloroacetate, trifluoroacetate, phosphate, glutamate, bicarbonate, para-toluenesulfonate, and undecanoate, but is not limited thereto. In addition, examples of acids that may be used to form acid addition salts include inorganic acids such as hydrochloric acid, hydrobromic acid, sulfuric acid, and phosphoric acid, and organic acids such as oxalic acid, maleic acid, succinic acid, and citric acid, but are not limited thereto.

The peptide may further include a targeting sequence, a tag, a labeled residue, an amino acid sequence designed to increase the half-life or stability of the peptide, may be conjugated with an antibody or antibody fragment, human serum albumin (HSA), etc. to enhance targeting, efficacy or stability, or may be modified at its N-terminus or C-terminus.

In the present invention, peptides synthesized through known techniques may be used. The method of synthesizing a peptide may be a chemical method or a biological method, and the chemical method may include, for example, a solution phase method; solid-phase methods including the tert-butyloxycarbonyl (Boc)/benzyl (Bzl) strategy and the 9-fluorenylmethoxycarbonyl (Fmoc)/tert-butyl (t-Bu) strategy (Kent S B H, Mitchell A R, Engelhard M, Merrifield R B (1979) Mechanisms and prevention of trifluoroacetylation in solid-phase peptide synthesis. Proc Natl Acad Sci USA 76(5):2180-2184); a method of immobilizing a first amino acid on a resin and extending the peptide chain in sequence order; or a method using microwaves, and the biological method may be a method using microorganisms, but is not limited thereto.

The term “fragment” as applied to polynucleotide or polypeptide sequences refers to a nucleic acid or peptide sequence of reduced length compared to the above-mentioned nucleic acid or protein, and includes at least a common portion of a nucleotide sequence or peptide sequence identical to the above-mentioned nucleic acid or protein. These nucleic acid fragments and polypeptide fragments according to the present invention may, where appropriate, be incorporated into larger polynucleotides or polypeptides as components. Such fragments include or consist of oligonucleotides or oligopeptides with a length of at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000, 1500, 2000, 3000, 4000, 5000 consecutive nucleotides or peptides of the nucleic acid or protein of the present invention.

A “variant” of a polypeptide or protein refers to any analog, fragment, derivative or mutant derived from the polypeptide or protein and retaining at least one biological property of the polypeptide or protein. Different variants of the polypeptide or protein may be present in nature. These variants may be allelic variations characterized by differences in the nucleotide sequence of the structural gene encoding proteins, or may include differential splicing or post-translational modifications. One of ordinary skill in the art may produce variants having one or more amino acid substitutions, deletions, additions or replacements. These variants include, among others: (a) variants in which one or more amino acid residues are substituted with conservative or non-conservative amino acids; (b) variants in which one or more amino acids are added to a polypeptide or protein; (c) variants in which one or more amino acids include a substituent, and (d) variants in which the polypeptide or protein is fused to another polypeptide such as serum albumin.

In addition, conservative variants refer to amino acid sequences with sequence alterations that do not adversely affect the biological function of the protein. When an altered sequence interferes with or destroys the biological function associated with the protein, a substitution, insertion, or deletion is described as adversely affecting a protein. For example, the overall charge, structure, or hydrophobicity-hydrophilicity of a protein may be altered without adversely affecting its biological activity. Therefore, an amino acid sequence may be altered, for example, to allow the peptide to exhibit higher hydrophobicity or hydrophilicity without adversely affecting the biological activity of the protein. Techniques for obtaining such variants are known to those skilled in the art, including genetic (inhibition, deletion, mutation, etc.), chemical, and enzymatic techniques.

A pharmaceutical composition for treating cancer, including a platycoside described herein, for example, platycodin D, platycodin D2, or polygalicin D, may be administered in the form of any suitable pharmaceutical composition that may include a pharmaceutically acceptable carrier and may optionally include one or more adjuvants, stabilizers, and the like. In one embodiment, the pharmaceutical composition is for use in therapeutic or prophylactic treatment, for example, treating or preventing cancer or tumors such as those described herein.

The term “pharmaceutical composition” relates to a formulation including a therapeutically effective substance, preferably together with pharmaceutically acceptable carriers, diluents and/or excipients. The pharmaceutical composition is useful for reducing the severity of, preventing, or treating a disease or disorder by administering the pharmaceutical composition to a subject. The pharmaceutical composition is also known in the art as a pharmaceutical formulation. In the context of the present invention, the pharmaceutical composition includes a peptide, protein, or polypeptide for delivering liposomes specifically to cancer cells or cancer tissues, as described herein. In the present invention, an ATRAM peptide was used as a peptide, protein, or polypeptide.

The pharmaceutical composition of the present invention may contain one or more adjuvants or may be administered together with one or more adjuvants. The term “adjuvant” refers to a compound that prolongs, enhances, or accelerates an immune response. Adjuvants include a heterogeneous group of compounds such as oil emulsions (e.g., Freund's adjuvant), mineral compounds (e.g., alum), bacterial products (e.g. pertussis toxin) or immune-stimulating complexes. Non-limiting examples of adjuvants include lipopolysaccharide (LPS), GP96, CpG oligodeoxynucleotides, growth factors, and cytokines such as monokines, lymphokines, interleukins, and chemokines. A cytokine may be IL1, IL2, IL3, IL4, IL5, IL6, IL7, IL8, IL9, IL10, IL12, IFNα, IFNγ, GM-CSF, or LT-a. Additional known adjuvants are aluminum hydroxide, Freund's adjuvant or oils such as Montanide® ISA51.

The pharmaceutical composition according to the present invention is generally applied in a “pharmaceutically effective amount” and as a “pharmaceutically acceptable formulation.”

The term “pharmaceutically acceptable” refers to the non-toxicity of a substance that does not interact with the action of an active ingredient of a pharmaceutical composition.

The term “pharmaceutically effective amount” or “therapeutically effective amount” means an amount that, alone or in combination with additional administration, achieves the desired response or desired effect. When treating a specific disease, the desired response preferably means inhibition of the progression of the disease. This includes slowing the progression of the disease, and in particular stopping or reversing the progression of the disease. The desired response in the treatment of a disease may also be delaying the onset or preventing the onset of the disease or condition. The effective dose of the composition described herein will be determined according to the condition being treated, the severity of the disease, the individual characteristics of the patient, including age, physical condition, height, and weight, the duration of treatment, the type of concomitant therapy (if any), the specific route of administration, and similar factors. Therefore, the dosage of the composition described herein may be determined according to these various characteristics. When the patient does not respond sufficiently to the first dose, a higher dose (or a higher dose achieved by another, more local route of administration) may be used.

The pharmaceutical composition of the present invention may include salts, buffers, preservatives, and optionally other therapeutic agents. In one embodiment, the pharmaceutical composition of the present invention includes one or more pharmaceutically acceptable carriers, diluents and/or excipients.

Preservatives suitable for use in the pharmaceutical composition of the present invention may include, but are not limited to, benzalkonium chloride, chlorobutanol, parabens, and thimerosal.

As used herein, the term “excipient” refers to a substance that may be present in the pharmaceutical composition of the present invention but is not an active ingredient. Non-limiting examples of excipients include carriers, binders, diluents, lubricants, thickeners, surfactants, preservatives, stabilizers, emulsifiers, buffers, flavoring agents, or colorants.

The term “diluent” refers to a diluting and/or thinning substance. In addition, the term “diluent” includes any one or more of fluids, liquids or solid suspensions, and/or mixed media. Examples of suitable diluents include ethanol, glycerol, and water.

The term “carrier” refers to an ingredient, which may be natural, synthetic, organic, or inorganic, that is combined with an active ingredient to facilitate, enhance, or enable administration of a pharmaceutical composition. As used herein, a carrier may be one or more compatible solid or liquid fillers, diluents, or encapsulating materials suitable for administration to a subject. Suitable carriers include, but are limited to, sterile water, Ringer's solution, Ringer's lactate, sterile sodium chloride solutions, isotonic saline, polyalkylene glycols, hydrogenated naphthalene, and, especially, biocompatible lactide polymers, lactide/glycolide copolymers or polyoxyethylene/polyoxypropylene copolymers, lipid membranes, lipid bilayers, micelles, or liposomes. In one embodiment, the pharmaceutical composition of the present invention includes isotonic saline.

Pharmaceutically acceptable carriers, excipients, or diluents for therapeutic use are well known in the pharmaceutical field and are described, for example, in Remington's Pharmaceutical Sciences, Mack Publishing Co. (A. R Gennaro edit. 1985).

Pharmaceutical carriers, excipients, or diluents may be selected according to the intended route of administration and standard pharmaceutical practices.

In one embodiment, the pharmaceutical composition described herein may be administered intravenously, intraarterially, subcutaneously, intradermally, or intramuscularly. In a specific embodiment, the pharmaceutical composition is formulated for topical or systemic administration. Systemic administration may include enteral administration, accompanied by absorption through the gastrointestinal tract, or parenteral administration. As used herein, “parenteral administration” refers to administration by any means other than via the gastrointestinal tract, such as by intravenous injection. In a preferred embodiment, the pharmaceutical composition is formulated for systemic administration. In another preferred embodiment, systemic administration is by intravenous administration. In one embodiment of all aspects of the present invention, the liposome including platycodin D2 or the pharmaceutical composition including the same described herein is administered systemically.

As used herein, the term “co-administration” refers to administering multiple compounds or compositions to the same patient. The multiple compounds or compositions may be administered simultaneously, essentially simultaneously, or sequentially.

The materials, compositions, and methods described herein may be used to treat a subject with a disease, for example, a disease characterized by the presence of diseased cells that express an antigen. A particularly preferred disease is cancer or a tumor. For example, when the antigen is derived from a virus, the materials, compositions, and methods may be useful in the treatment of a viral disease caused by the virus.

The term “cancer disease” or “cancer” refers to or means a pathological condition in a subject that is typically characterized by uncontrolled cell proliferation. Non-limiting examples of cancer include carcinomas, lymphomas, blastomas, sarcomas, and leukemia. More specifically, the present invention is for treating solid cancer, and examples of such cancer include lung cancer, liver cancer, pancreatic cancer, skin cancer, head and neck cancer, skin or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's disease, esophageal cancer, small intestine cancer, endocrine cancer, thyroid cancer, parathyroid cancer, adrenal cancer, soft tissue sarcoma, bladder cancer, kidney cancer, renal cell carcinoma, renal pelvis carcinoma, neoplasms of the central nervous system (CNS), neuroectodermal cancer, spinal tumors, gliomas, meningiomas, and pituitary adenomas. In one embodiment of the present invention, the cancer to be treated is CRC, and according to the present specification, the term “cancer” also includes metastatic cancer. In one embodiment of the present invention, the pharmaceutical composition of the present invention was confirmed to have a therapeutic effect on CRC, breast cancer, or lung cancer, and among them, it was confirmed that the pharmaceutical composition had an excellent therapeutic effect on CRC.

Combination strategies in cancer treatment may be suitable due to the synergistic effects achieved, which may be considered higher than the effectiveness of monotherapy approaches. In one embodiment, the pharmaceutical composition is administered together with an immunotherapeutic agent. As used herein, “immunotherapeutic agent” refers to any material that can be involved in activating a specific immune response and/or immune effector function(s). The present specification contemplates the use of antibodies as an immunotherapeutic agent. Without wishing to be bound by theory, antibodies may achieve their therapeutic effects on cancer cells through a variety of mechanisms, including inducing apoptosis, blocking components of signaling pathways, or inhibiting proliferation of tumor cells. In a specific embodiment, the antibody is a monoclonal antibody. Monoclonal antibodies may induce cell death through antibody-dependent cell-mediated cytotoxicity (ADCC), or they may bind to complement proteins and induce direct cytotoxicity, known as complement-dependent cytotoxicity (CDC). Non-limiting examples of anti-cancer antibodies and potential antibody targets (in parentheses) that can be used in combination with the present invention include abagovomab (CA-125), abciximab (CD41), adecatumumab (atumumab) (EpCAM), afutuzumab (CD20), alacizumab pegol (VEGFR2), altumomab pentetate (CEA), amatuximab (MORAb-009), anatumomab mafenatox (TAG-72), apolizumab (HLA-DR), arcitumomab (CEA), atezolizumab (PD-L1), bavituximab (phosphatidylserine), bectumomab (CD22), belimumab (BAFF), bevacizumab (VEGF-A), bivatuzumab mertansine (CD44 v6), blinatumomab (CD19), brentuximab vedotin (CD30 TNFRSF8), cantuzumab mertansin (mucin CanAg), cantuzumab ravtansine (MUC1), caproumab pendetide (prostate carcinoma cells), carlumab (CNT0888), catumaxomab (EpCAM, CD3), cetuximab (EGFR), citatuzumab bogatox (EpCAM), cixutumumab (IGF-1 receptor), claudiximab (Claudin), clivatuzumab tetraxetan (MUC1), conatumumab (TRAIL-R2), dacetuzumab (CD40), dalotuzumab (insulin-like growth factor I receptor), denosumab (RANKL), detumomab (B-lymphoma cells), drozitumab (DR5), ecromeximab (GD3 ganglioside), edrecolomab (EpCAM), elotuzumab (SLAMF7), enavatuzumab (PDL192), ensituximab (NPC-1C), epratuzumab (CD22), ertumaxomab (HER2/neu, CD3), etaracizumab (Integrin αγβ3), farletuzumab (folate Receptor 1), FBTA05 (CD20), ficlatuzumab (SCH 900105), figitumumab (IGF-1 receptor), flanvotumab (glycoprotein 75), fresolimumab (TGF-β), galiximab (CD80), ganitumab (IGF-I), gemtuzumab ozogamicin (CD33), gevokizumab (IL1β), girentuximab (carbonic anhydrase 9 (CA-IX)), glembatumumab vedotin (GPNMB), ibritumomab tiuxetan (CD20), icrucumab (VEGFR-1), igovoma (CA-125), indatuximab ravtansine (SDC1), intetumumab (CD51), inotuzumab ozogamicin (CD22), ipilimumab (CD 152), iratumumab (CD30), labetuzumab (CEA), lexatumumab (TRAIL-R2), libivirumab (hepatitis B surface antigen), lintuzumab (CD33), lorvotuzumab mertansine (CD56), lucatumumab (CD40), lumiliximab (CD23), mapatumumab (TRAIL-R1), matuzumab (EGFR), mepolizumab (IL5), milatuzumab (CD74), mitumomab (GD3 ganglioside), mogamulizumab (CCR4), moxetumomab pasudotox (CD22), nacolomab tafenatox (C242 antigen), naptumomab estafenatox (5T4), namatumab (RON), necitumumab (EGFR), nimotuzumab (EGFR), nivolumab (IgG4), ofatumumab (CD20), olaratumumab (PDGF-R a), onartuzumab (human scatter factor receptor kinase), oportuzumab monatox (EpCAM), oregovomab (CA-125), oxelumab (OX-40), panitumumab (EGFR), patritumab (HER3), pemtumoma (MUC1), pertuzuma (HER2/neu), pintumomab (adenocarcinoma antigen), pritumumab (vimentin), racotumomab (N-glycolylneuraminic acid), radretumab (fibronectin extra domain-B), rafivirumab (rabies virus glycoprotein), ramucirumab (VEGFR2), rilotumumab (HGF), rituximab (CD20), robatumumab (IGF-1 receptor), samalizumab (CD200), sibrotuzumab (FAP), siltuximab (IL6), tabalumab (BAFF), tacatuzumab tetraxetan (α-fetoprotein), taplitumomab paptox (CD 19), tenatumomab (Tenascin C), teprotumumab (CD221), ticilimumab (CTLA-4), tigatuzumab (TRAIL-R2), TNX-650 (IL13), tositumomab (CD20), trastuzumab (HER2/neu), tRBS07 (GD2), tremelimumab (CTLA-4), tucotuzumab celmoleukin (EpCAM), ublituximab (MS4A1), urelumab (4-1 BB), volociximab (integrin α5β1), votumumab (tumor antigen CTAA 16.88), zalutumumab (EGFR), and zanolimumab (CD4).

The materials, compositions, and methods described herein may be used in the therapeutic or prophylactic treatment of a variety of diseases. In one embodiment, the materials, compositions, and methods described herein are useful for the prophylactic and/or therapeutic treatment of cancer or diseases associated with tumors.

The term “disease” refers to an abnormal condition affecting an individual's body. Diseases are often interpreted as a medical condition that is associated with specific symptoms and signals. A disease may be caused by factors originating from an external source, such as an infectious disease, or may be caused by internal dysfunction, such as an autoimmune disease. In humans, “disease” is used in a broader sense to refer to any condition in which an illness, upon contact with an individual, causes pain, dysfunction, suffering, social problems, death, or similar problems in the afflicted individual. In a broader sense, it sometimes includes injuries, disabilities, impairments, syndromes, infections, isolated symptoms, aberrant behavior, and structural and functional atypical deformities, which may be considered distinct categories in other contexts and for other purposes. Diseases generally affect individuals not only physically but also emotionally, as living with various diseases may change an individual's perspective on life and personality.

In this context, the terms “treatment,” “treating,” or “therapeutic intervention” mean the management and care of an individual for the purpose of combating a condition such as a disease or disorder. These terms are intended to include alleviating symptoms or complications, and/or delaying the progression of a disease, disorder or condition, and/or alleviating or mitigating symptoms and complications, and/or curing or eliminating a disease, disorder or condition, as well as the full spectrum of treatment for a given condition suffered by an individual for preventing the condition, such as the administration of a therapeutically effective compound, wherein prevention is understood as the management and care of an individual for the purpose of combating the disease, condition or disorder and includes the administration of an active compound to prevent the onset of symptoms or complications.

The term “therapeutic treatment” refers to any treatment that improves the health status of an individual and/or prolongs (increases) the lifespan of an individual. The treatment may be eliminating a disease in an individual, stopping or slowing down the progression of a disease in an individual, inhibiting or slowing down the progression of a disease in an individual, reducing the frequency or severity of symptoms in an individual, and/or reducing recurrence in an individual who is currently suffering from or who has previously suffered from a disease.

The term “prophylactic treatment” or “preventative treatment” refers to any treatment intended to prevent a disease from developing in an individual. The terms “prophylactic treatment” and “preventative treatment” are used interchangeably herein.

The terms “individual” and “subject” are used interchangeably herein. These terms refer to humans or other mammals (e.g., mice, rats, rabbits, dogs, cats, cows, pigs, sheep, horses, or primates) that may be susceptible to or vulnerable to a disease or disorder (e.g., cancer), but they may or may not have a disease or disorder. In many embodiments, the subject is a human. Unless otherwise specified, the terms “individual” and “subject” do not refer to a specific age and thus encompass adults, the elderly, children, and newborns. In embodiments herein, the “individual” or “subject” is a “patient.”

The term “patient” refers to an individual or subject in need of treatment, specifically an individual or subject with a disease.

The health functional food of the present invention may be prepared and processed in the form of tablets, capsules, powder, granules, liquids, pills, and the like for anti-cancer purposes.

The health functional food of the present invention refers to food that is prepared and processed using raw materials or ingredients having functionality useful to the human body in accordance with Health Functional Food Act No. 6727 and ingested for the purpose of regulating nutrients for the structure and functions of the human body or obtaining useful effects for healthcare purposes such as physiological actions.

The health functional food of the present invention may contain common food additives, and their suitability as a food additive is determined in accordance with the specifications and standards for the items according to the general provisions of the Food Additive Code approved by the Food and Drug Administration and general test methods, unless otherwise specified.

The items listed in the Food Additives Code include, for example, chemical compounds such as ketones, glycine, calcium citrate, nicotinic acid, and cinnamic acid; natural additives such as persimmon color, licorice extract, crystalline cellulose, Kaoliang color, and guar gum; and mixed preparations such as sodium L-glutamate preparations, noodle-added alkaline preparations, preservative preparations, and tar coloring preparations, but are not limited thereto.

For example, for manufacture of health functional food in the form of tablets, a mixture prepared by mixing the peptide with an excipient, a binder, a disintegrant, and other additives is granulated by a conventional method and then molded by compression after adding a lubricant, or the mixture is directly molded by compression. In addition, the health functional food in the form of tablets may contain a flavoring agent and the like as needed.

Among the health functional food in the form of capsules, hard capsules may be manufactured by filling common hard capsules with a mixture prepared by mixing the peptide with additives such as excipients, and soft capsules may be manufactured by filling a mixture prepared by mixing the peptide with additives such as excipients into a capsule base such as gelatin. The soft capsules may contain plasticizers such as glycerin or sorbitol, colorants, preservatives, and the like as needed.

The health functional food in the form of pills may be prepared by molding a mixture prepared by mixing the peptide with an excipient, a binder, a disintegrant, and the like by a known method, and it may be coated with white sugar or another coating agent or the surface may be coated with a material such as starch and talc as needed.

The health functional food in the form of granules may be manufactured into granules by mixing the peptide with an excipient, a binder, a disintegrant, and the like by a conventional method, and it may contain a flavoring agent, a flavor enhancer, etc. as needed.

The health functional food includes beverages, meat, chocolate, food, confectioneries, pizza, ramen, other noodles, gum, candies, ice cream, alcoholic beverages, vitamin supplements, and health supplements.

### EXAMPLES

### Example 1. Materials and Methods

### 1.1. Materials

Platycodin D (PCD, molecular weight (MW)=1225.34, purity=98%), platycodin D2 (PCD2, MW=1387.48, purity=97%), polygalacin D (PGD, MW=1209.34, purity=98%) were purchased from Biopurify Phytochemicals Ltd. (China). An ATRAM peptide (GLAGLAGLLGLEGLLGLPLGLLEGLWLGLELEGN) (SEQ ID NO: 1) was purchased from PEPTRON Inc. (Korea). Dulbecco's modified Eagle's medium (DMEM) with high glucose with L-glutamine, RPMI-1640 media with L-glutamine, fetal bovine serum (FBS), streptomycin, penicillin, and 0.25% trypsin-EDTA were purchased from GenDEPOT (USA). A Quanti-LDH™ PLUS Cytotoxicity Assay Kit (Colorimetric) and a Quanti-Max™ WST-8 Cell Viability Assay Kit were purchased from BIOMAX (Korea). A MMR SPARK® microplate reader was purchased from TECAN Ltd. (Switzerland). Phosphatidylcholine was supplied by Solus Advanced Materials Co. Ltd. (Korea). Anhydrous methylene chloride, pyridine, N,N-diisopropylethylamine, 4-nitrophenylchloroformate, N-(2-aminoethyl) maleimide trifluoroacetate salt, and IR-783 were purchased from Sigma-Aldrich (USA), and MEL was supplied by DK Bio Co. Ltd. (Korea). PEO-b-PCL-b-PEO (MW˜20,000 gmol⁻¹, MW of blocks 5000-10000-5000 gmol⁻¹) was supplied by Hyundai Bioland Co. Ltd. (Korea). Texas Red-DHPE (DHPE; 1,2-dihexadecanoyl-sn-glycero-3-phosphoethanolamine, triethylammonium salt) and 4′,6-diamidino-2-phenylindole (DAPI) was purchased from Thermo Fisher Scientific (USA), and a phosphate-buffered saline (PBS) solution was purchased from GIBCO-Thermo Fischer Scientific (USA).

### 1.2. Cell Culture

Primary bone marrow-derived macrophages (BMDMs) were prepared by extraction from female C57BL/6 mice and then differentiated for 5 to 7 days in a medium containing M-CSF. Raw 264.7, DLD-1, CT26, HCT 116, A549, and MB231 cells were purchased from Korean Cell Line Bank (KCLB). BMDMs and Raw 264.7 cells were cultured in DMEM containing 10% FBS, streptomycin (100 μg/ml), and penicillin (100 U/ml). DLD-1, CT26, HCT116, A549, and MB 231 cells were cultured in RPMI 1640 containing 10% FBS, streptomycin (100 μg/ml), and penicillin (100 U/ml).

### 1.3. Animals

Female BALB/c nude mice (6 weeks old) were provided by Orient Bio Inc. (Korea), and wild-type female C57BL/6 mice were provided by Samtako Bio Korea Co. Ltd. (Republic of Korea) and maintained under standard housing conditions at the Center for Laboratory Animal Science, Hanyang University (Ansan, Korea). All animal breeding and experiments were carried out in accordance with the guidelines approved by the Institutional Animal Care and Use Committee (IACUC) of Hanyang University (Protocols 2021-0124 and 2021-0271).

### 1.4. Fabrication and Characterization of PS-Lipo

The present inventors fabricated 0.5 wt % liposomes using three kinds of platycosides (PS): PCD-loaded liposomes (PCD-Lipo), PCD2-loaded liposomes (PCD2-Lipo), and PGD-loaded liposomes (PGD-Lipo). For this, the present inventors dissolved phosphatidyl choline and PEO-b-PCL-b-PEO in chloroform and a platycoside in methanol, and then mixed the two solutions together in a round-bottomed flask. The mixing ratio of phosphatidylcholine and PEO-b-PCL-b-PEO was adjusted to 9:1, and the concentration of PS was set to 200 μM. For the peptide conjugation, MEL-maleimide (0.23 mgmL⁻¹) synthesized according to a known method was added to the mixture of amphiphiles. Subsequently, the solvent was evaporated using a rotary evaporator at 40° C. for one hour to form a thin film on the bottom of the round-bottomed flask. Then, PBS was added to the flask, and the film was hydrated in a sonicator at 45° C. for 100 min. To conjugate the ATRAM peptide with the MEL-malemide linker co-assembled with a vesicular membrane, ATRAM-cysteamide was added to the liposome dispersion, and the resulting mixture was stirred at 4° C. overnight. The hydrodynamic particle size of PS-Lipo and C-Lipo was measured by dynamic light scattering (DLS 1070, Malvern, UK) at 25° C. The morphology of PS-Lipo and C-Lipo was observed using a transmission electron microscope (JEM 1010, JEOL, Japan).

### 1.5. Cellular Uptake of PS-Lipo

To observe the in vitro cellular uptake behavior of PS-Lipo, BMDMs, Raw 264.7, DLD-1, and CT26 cells were plated at a density of 5×10³ cells per well in each 96-well plate (SPL, Korea) and cultured and maintained at 37° C. and 5% CO₂ for 24 hrs. Then, the cells were treated with 5 μM of C-Lipo, PCD-Lipo, PCD2-Lipo and PGD-Lipo containing Texas Red dye. After the designated time, the sample solution was discarded, the cells were washed twice with sterilized PBS, and the fluorescence intensity was measured in the range of 535 nm to 595 nm using a colorimetric microplate reader.

### 1.6. Confocal Microscopy

To observe the in vitro cellular uptake behavior of PS-Lipo, DLD-1 and CT26 cells were plated at a density of 2×10⁴ cells per well into each 12-well plate (SPL, Korea) with cover slides and cultured under the conditions of 37° C. and 5% CO₂. Both cells were treated with PS-Lipo and C-Lipo containing Texas Red dye (5 μM in serum-free RPMI-1640) for one hour. Subsequently, the cover slides were washed three times with sterile PBS, and the cells were fixed with 4% paraformaldehyde. After staining the nuclei of the cells with 4′,6-diamidino-2-phenylindole (DAPI) for 10 min, the cells were observed using a confocal microscope (LSM 800; Carl Zeiss, Germany).

### 1.7. In Vitro Cell Viability and Cytotoxicity Study

BMDMs, Raw 264.7, DLD-1, CT26, HCT 116, A549, and MB231 cells were plated into 96-well plates (SPL, Korea) at a density of 1×10⁴ cells per well and cultured in complete DMEM and RPMI-1640 for 18 h. Then, the cells were treated with free-PCD, free-PCD2, free-PGD, PCD-Lipo, PCD2-Lipo and PGD-Lipo at a series of concentrations. After incubation for 48 h, the medium was replaced with 100 μl of Quanti-Max solution (10% of each media volume). In addition, the cell culture supernatant was treated with 10 μl of the Quanti-LDH reaction mixture. After incubation for two hours at 37° C. and 5% CO₂, the absorbance of the plates treated with the Quanti-Max solution were measured at 450 nm using a colorimetric microplate reader. In addition, after incubation for 30 min at 37° C. and 5% CO₂, the absorbance of the plates treated with the Quanti-LDH reaction mixture was measured at 490 nm using a colorimetric microplate reader. Cell viability was calculated using untreated cells as a control, and cell cytotoxicity was calculated using, as a control, cells that were killed when a cell lysis solution was used.

### 1.8. Immunoblotting

For immunoblotting, cells were lysed with a radioimmunoprecipitation (RIPA) cell lysis buffer containing EDTA (GenDEPOT, USA) and a protease inhibitor cocktail (Xpert Protease Inhibitor Cocktail Solution; GenDEPOT, USA). The cell lysate was incubated at 4° C. for 30 min, and then centrifuged at 12,000 g for 10 min at 4° C. The proteins were analyzed by sodium dodecyl sulfate-polyacrylamide gel electrophoresis (SDS-PAGE) and transferred to a polyvinylidene difluoride (PVDF) membrane (GenDEPOT, USA). Anti-BCL-2 (3498S, Cell Signaling Technology (CST) Inc., USA), Anti-BCL-XL (2764S, CST, USA), Anti-Mcl-1 (5453S, CST, USA), Anti-PARP (9532S, CST, USA), Anti-Cleaved PARP (94885S, CST, USA), Anti-Cleaved Caspase-9 (9508S, CST, USA), Anti-Cleaved Caspase-9 (52873S, CST, USA), Anti-Caspase-3 (9662S, CST, USA), Anti-Cleaved Caspase-3 (9664S, CST, USA), Anti-Caspase-8 (4790S, CST, USA), Anti-GAPDH (sc-32233, Santa Cruz Biotechnology Inc., USA), and Anti-β-Actin (sc-47778, Santa Cruz Biotechnology Inc., USA) were incubated at 4° C. overnight, and the antibody conjugation was visualized by EzWestLumi plus (Atto Corporation, Japan) and detected by a Vilber chemiluminescence analyzer (Fusion Solo, Vilber Lourmat, France).

### 1.9. Generation of Knockout Cell Lines Using CRISPR/Cas9

For gene editing caspase-9, DLD-1 and CT26 cells were plated into 24-well plates (SPL, Korea) at a density of 5×10⁴ cells per well and cultured under the conditions of 37° C. and 5% CO₂ for 24 h. A mixture of a gRNA targeting caspase-9 (CRISPR995170_SGM and CRISPR31379_SGM, Invitrogen, USA), a Cas9 protein (TrueCut™ Cas9 Protein v2, Invitrogen, USA), and Lipofectamine (Lipofectamine™ CRISPRMAX™, Invitrogen, USA) was produced and cultured, and the culture was performed under the conditions of 37° C. and 5% CO₂ for two days. After a selection process using a genomic cleavage detection assay, knockout of caspase-9 was confirmed by Quantitative PCR and Western Blot analysis.

### 1.10. RNA Isolation and Quantitative PCR

Total RNA was isolated from DLD-1 and CT26 cells using the TRIzol™ reagent (Invitrogen, USA). Then, cDNA synthesis was carried out using 100 ng of the total RNA and a Moloney murine leukemia virus (M-MLV) cDNA synthesis kit (Enzynomics, Korea). Quantitative real-time (RT) PCR was carried out using the comparative cycle threshold (CT) method on the QuantStudio 3 Real-Time PCR Instrument (Applied Biosystems, USA). After pre-denaturation at 95° C. for 10 min, amplification was performed with 40 cycles of 95° C. for 15 s, 56° C. for 20 s, and 72° C. for 30 s. The human caspase-9 primer pair was 5′ CATTTCATGGTGGAGGTGAAG-3′ (SEQ ID NO: 2) and 5′-GGGAACTGCAGGTGGCTG-3′ (SEQ ID NO: 3). The human GAPDH primer pair was 5′-GGTGTGAACCATGAGAAGTATGA-3′ (SEQ ID NO: 4) and 5′-GAGTCCTTCCACGATACCAAAG-3′ (SEQ ID NO: 5). The mouse caspase-9 primer pair was 5′-AGTTCCCGGGTGCTGTCTAT-3′ (SEQ ID NO: 6) and 5′-GCCATGGTCTTTCTGCTCAC-3′ (SEQ ID NO: 7). The mouse GAPDH primer pair was 5′-ACTCCACTCACGGCAAATTC-3′ (SEQ ID NO: 8) and 5′-TCTCCATGGTGGTGAAGACA-3′ (SEQ ID NO: 9).

### 1.11. Preparation of Xenograft Mice Models

Female BALB/c nude mice were used for tumor xenograft experiments. To assess colorectal cancer-bearing mice, CT26 cells (1×10⁶ cells/100 μl/animal, n=5), CT26 caspase-9 K.O cells (1×10⁶ cells/100 μl/animal, n=5), DLD-1 cells (3×10⁶ cells/100 μl/animal, n=10), and DLD-1 caspase-9 K.O cells (3×10⁶ cells/100 μl/animal, n=10) were subcutaneously injected into the right axillary region. The animals were observed for 28 days with tumor volume measurements. Treatment was initiated when the tumor size reached an average volume of 50 or 200 mm³. The tumor volume was measured every day with skin calipers and calculated as (tumor length)×(tumor width)²×0.5, and expressed in mm³. All animals were maintained in a specific pathogen-free environment.

### 1.12. Histology and Immunohistochemistry of Tumor Tissues

Tumor tissues were fixed in neutral buffered formalin (pH 7.4) and embedded in paraffin. Tissue slides (4 μm) were stained with H&E, and immunohistochemical analysis was performed using the streptavidin-biotin and peroxidase protocol according to the manufacturer's instructions (Dako, Carpinteria, CA, USA) to detect Ki-67 (ab16667, ABCAM, USA) and proliferating cell nuclear antigen (PCNA, E-AB-32521, Elabscience, USA) proteins. The immunostaining content was semi-quantified using image analysis software (Image Pro Plus 4.5; Media Cybernetics Inc., Bethesda, MD, USA). Brown staining appeared under the optical microscope reflecting positive antigens, and a board-certified pathologist (Dr. Min-Kyung Kim, Kim Min-Kyung Pathology Clinic, Seoul, Korea) independently scored each slide without prior knowledge of the treatment groups.

### 1.13. In Vivo Biodistribution of PCD2-Lipo in Xenograft Mice Models

The in vivo biodistribution of PCD2-Lipo and PCD2-Lipo-ATRAM with IR783 in the xenograft mouse models established using the DLD-1 cells was observed via the In Vivo Imaging System (IVIS) Spectrum-CT in vivo imaging system (PerkinElmer, USA). PCD2-Lipo and PCD2-Lipo-ATRAM (2.5 mg kg-1, 100 μl) were intraperitoneally injected into the tumor-bearing mouse models (n=3 or 4/group). After injection, post-injection time-dependent whole body near-infrared fluorescence (NIRF) imaging was performed up to 48 h.

### 1.14. Statistical Analysis

All data was reported as mean±standard deviation (SD) or standard error of the mean (SEM) and was analyzed using the Student's t-test with a Bonferroni correction or analysis of variance (ANOVA) for multiple comparisons. Analysis was performed using the statistical software program SPSS (Version 12.0) (SPSS, Chicago, IL, USA).

### Example 2. Results

### 2.1. Preparation and Characterization of PS-Lipo

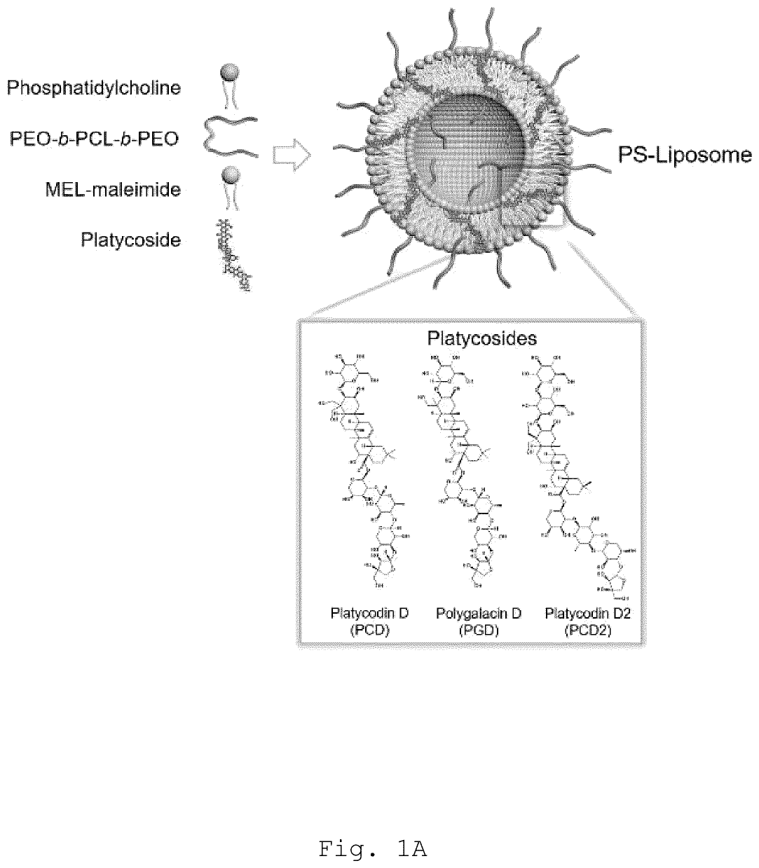

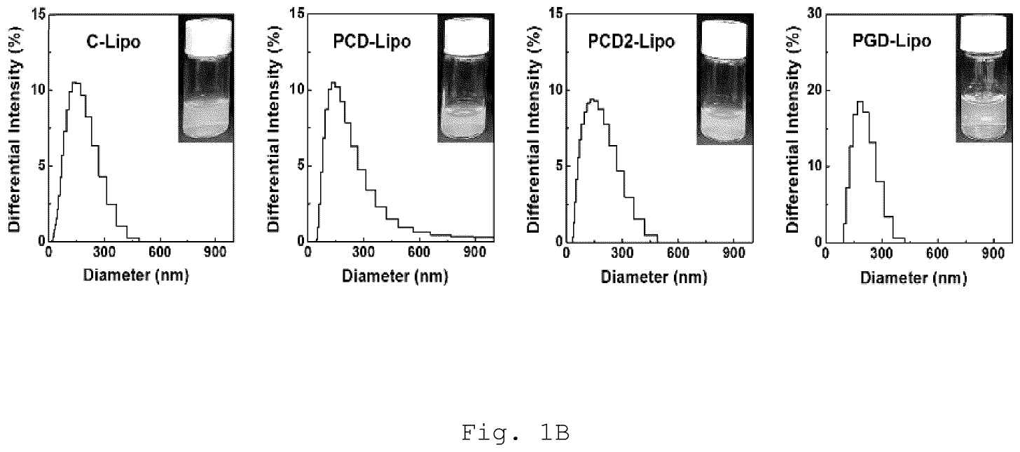

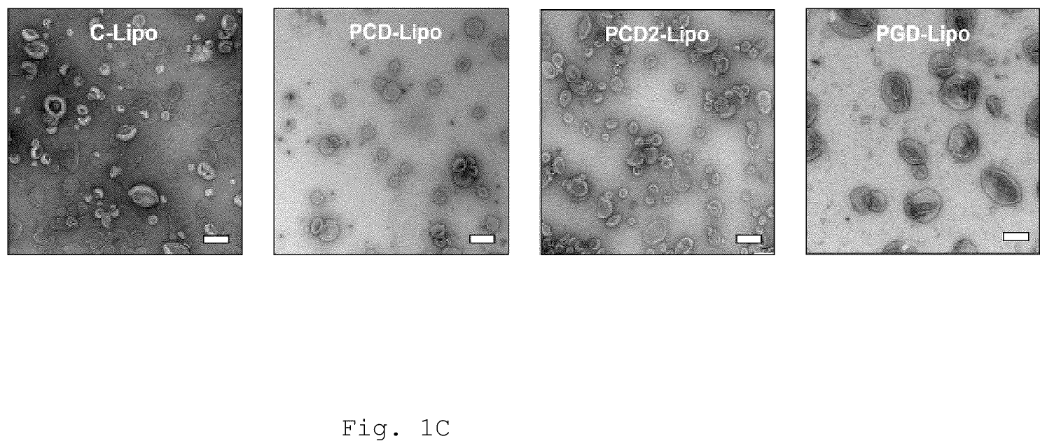

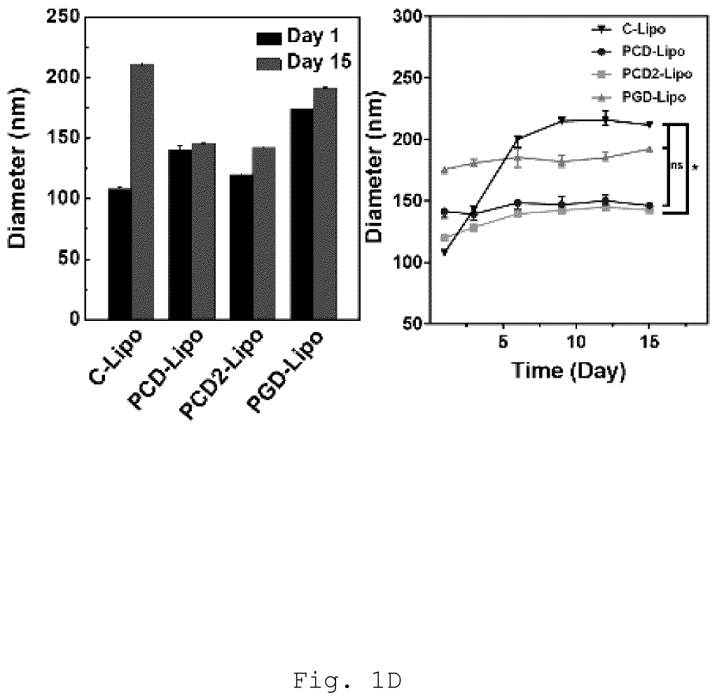

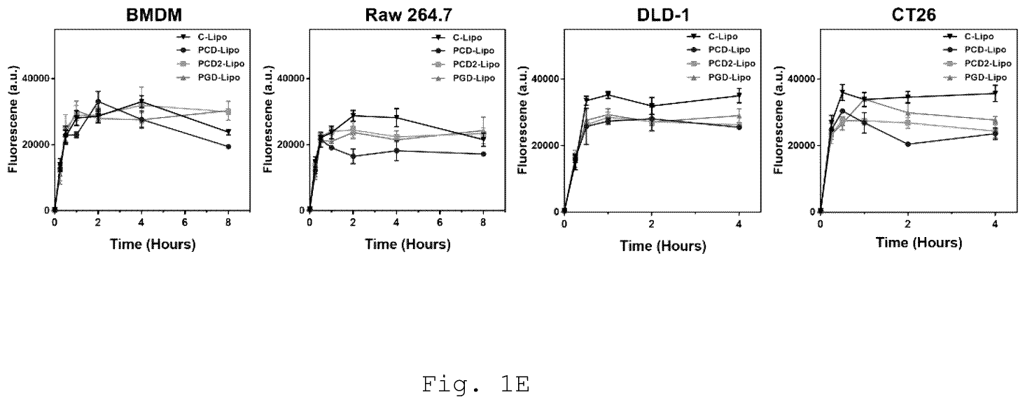

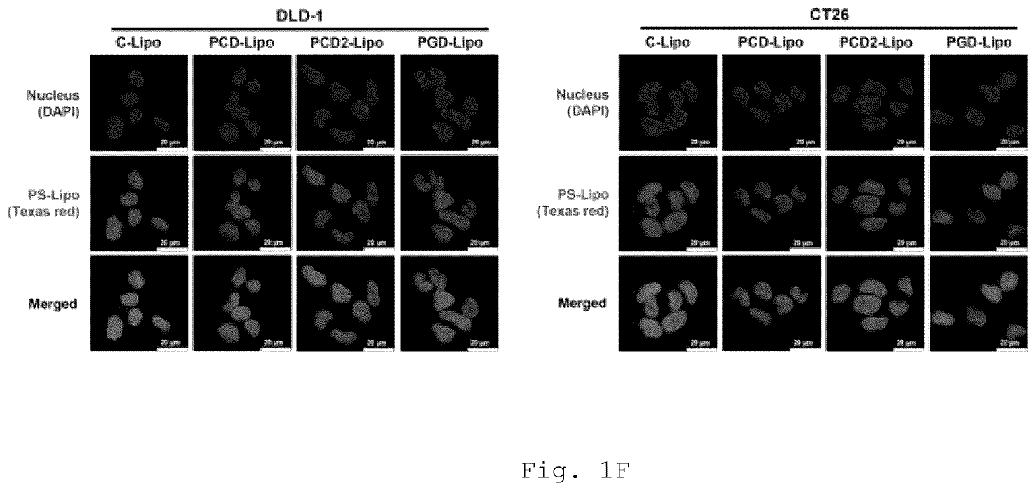

To overcome the disadvantages of limited delivery efficiency and poor physicochemical properties of PS, three different PS-Liposomes (PS-Lipo), namely PCD-Lipo, PCD2-Lipo, and PGD-Lipo, were fabricated (FIG. 1A). To induce PEGylation, an amphiphilic triblock copolymer, poly(ethylene oxide)-b-poly(ε-caprolactone)-b-poly-(ethylene oxide) (PEO-b-PCL-b-PEO), was laterally assembled along with the lipid bilayer of liposomes, thus enhancing the membrane modulus and systemic circulation time of liposomes.

In addition, liposomes were prepared by a thin-film hydration method to achieve high encapsulation efficiency and fine particle size distribution of liposomes. The typical vesicular structure of the control liposomes (C-Lipo) and PS-Lipo were confirmed by transmission electron microscopy (TEM) (FIG. 1B). The average hydrodynamic particle size of C-Lipo was 108.7 nm with a polydispersity index (PDI) of approximately 0.28. All PS-Lipo molecules had a slightly higher average hydrodynamic particle size compared to C-Lipo. The hydrodynamic sizes of PCD-Lipo, PCD2-Lipo, and PGD-Lipo were 139.9 nm, 120.0 nm, and 175.2 nm with the PDI values of 0.350, 0.357, and 0.159, respectively (FIG. 1C). When a long-term stability test was performed, negligible changes were observed in the particle sizes of the three PS-Lipo formulations (FIG. 1D). These results suggest that PS was physically and stably anchored to the lipid bilayers in the liposome systems and did not affect the liposome formation process and long-term storage. To determine the uptake efficiency of the prepared PS-Lipo molecules, macrophage cells (BMDM and Raw 264.7) and cancer cells (DLD-1 and CT26) were treated with Texas Red-DHPE-tagged PS-Lipo over time. All three PS-Lipo molecules exhibited strong red fluorescence signals inside different cells (FIGS. 1E and 1F), suggesting that PS encapsulated by liposomes could be successfully internalized by cells.

2.2. Encapsulating PS with Liposomes Enhances Cytotoxicity Against CRC Cells

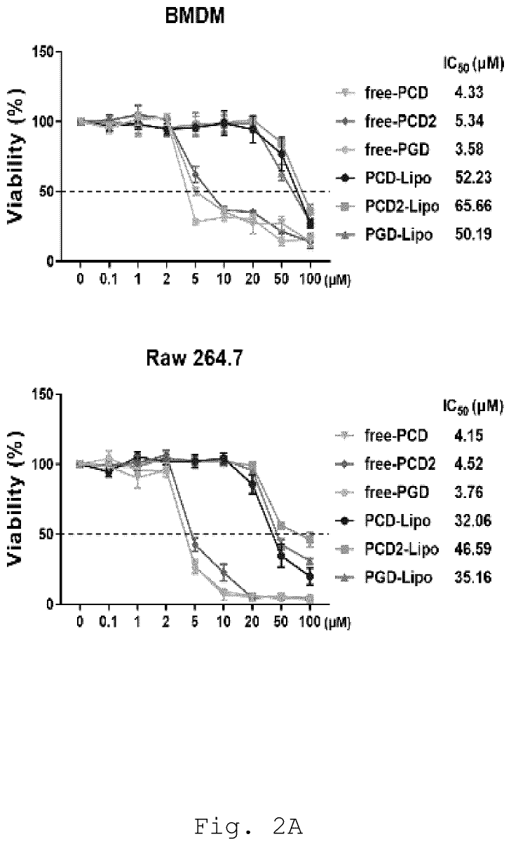

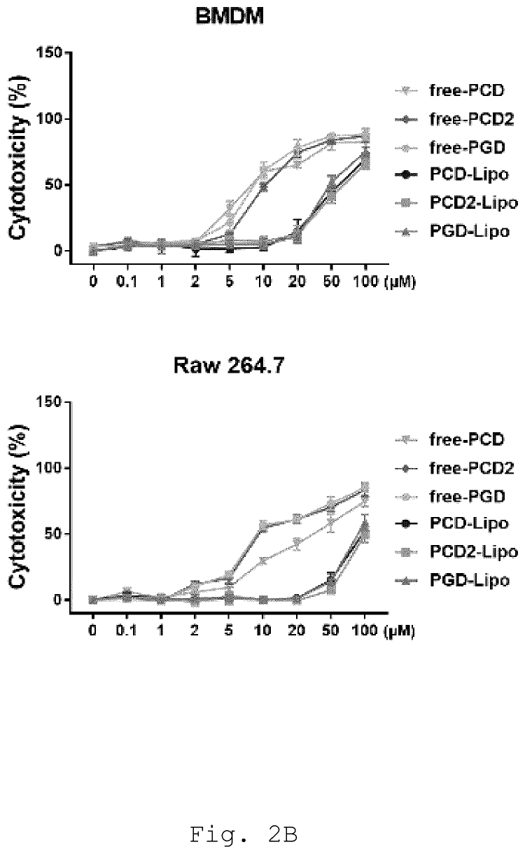

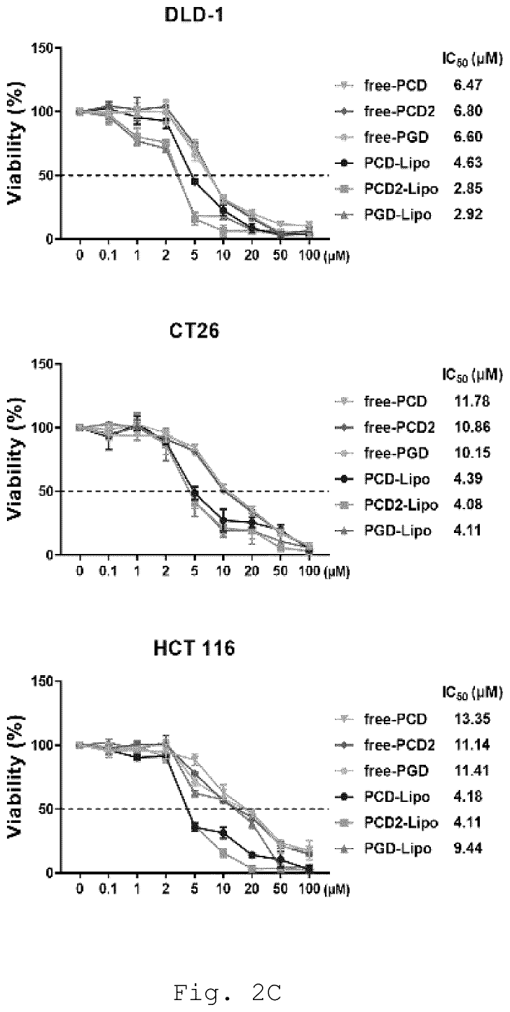

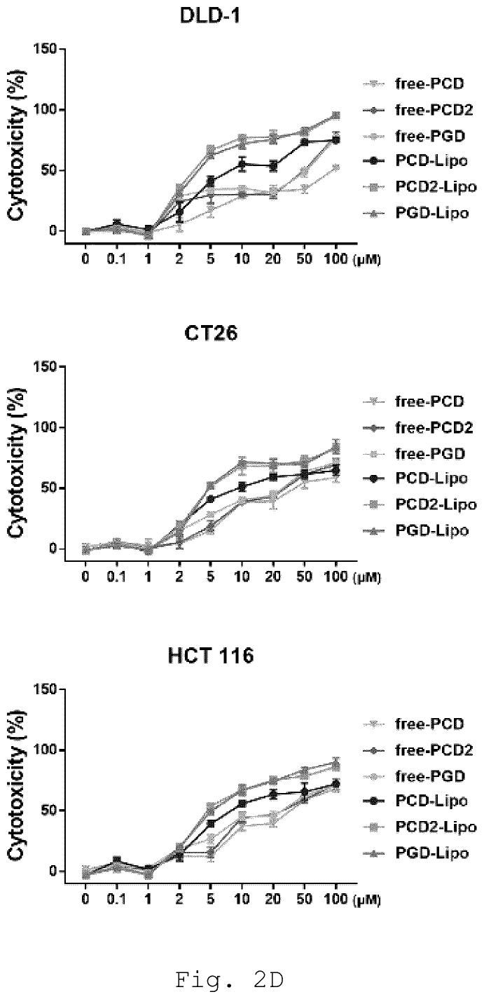

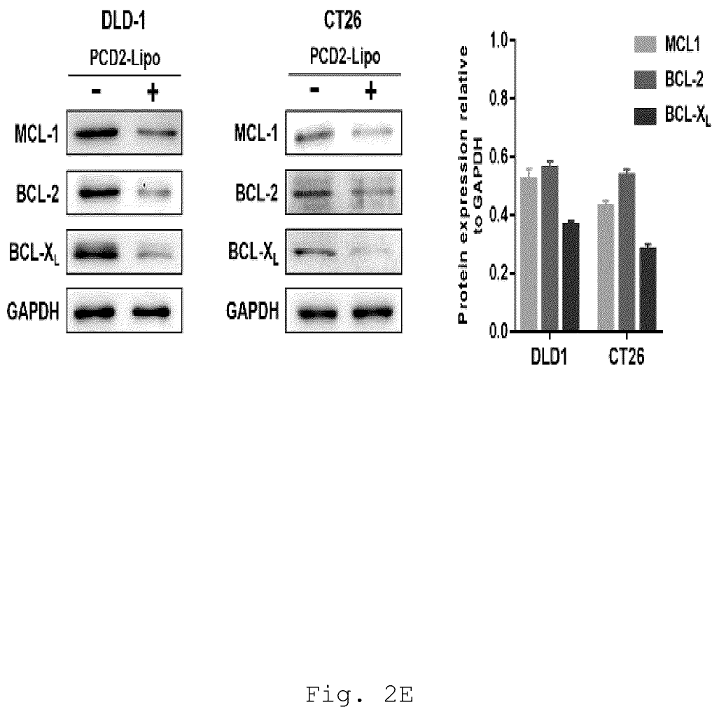

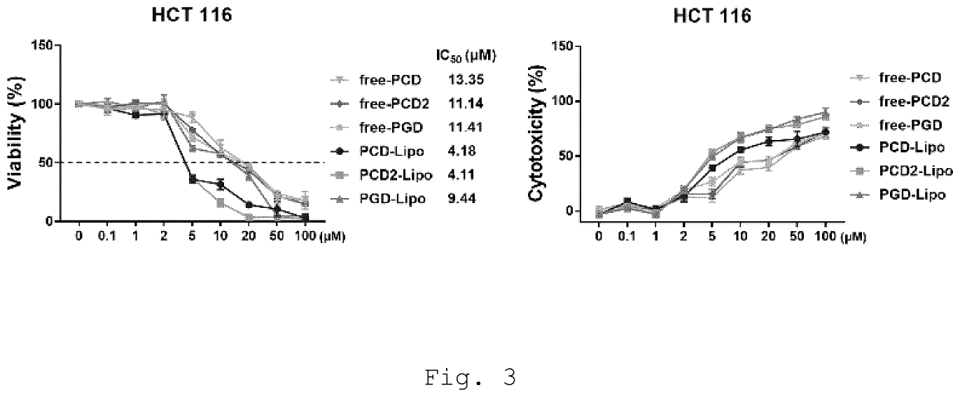

The antitumor activity of PS-Lipo samples was investigated through WST-8 cell viability and Quanti-LDH (lactate dehydrogenase)™ cytotoxicity assays. Different cancer cells were used in these assays, and macrophages were used as controls. First, the viability of macrophages was tested by treating them with free PS and PS-Lipo. As illustrated in FIGS. 2A and 2B, it was observed that free PCD, PCD2, and PGD significantly inhibited the proliferation of macrophages, most likely due to the instability of free platycosides in vitro. However, when free PS was encapsulated in liposomes (PS-Lipo), its toxicity to normal cells was substantially reduced, this is probably due to the increased stability and enhanced cellular uptake of free platycosides, indicating that the prepared PS-Lipo molecules possess remarkable biocompatibility at the cellular level. Next, the present inventors performed cytotoxicity assays against different cancer cell lines to evaluate the antitumor efficacy of PS-Lipo. The viability of colon, breast, and lung cancer cells was gradually inhibited by each PS-Lipo in a concentration-dependent manner (FIGS. 2C, 2D, and 3). Interestingly, each PS-Lipo exerted significant cytotoxicity on various cancer cell lines, with CRC cells being the most sensitive. Consistent with these viability results, the LDH assay showed that PS-Lipo treatment exhibited the strongest cytotoxicity against CRC cell lines (FIG. 2C). The cytotoxicity of the three types of PS-Lipo against CRC cells was in the order PCD2-Lipo>PGD-Lipo>PCD-Lipo (FIG. 2C).

Most cancer cells tend to upregulate the expression levels of the BCL-2 protein family, which is a typical feature of cancer. Among the antiapoptotic BCL-2 protein family, BCL-XL has been observed to be strongly upregulated in human CRC samples and it plays a crucial role in determining the chemoresistance of CRC cancer stem cells. A WB analysis was carried out using MCL-1, BCL-XL, and BCL-2 proteins to examine whether the stronger cytotoxicity of PS-Lipo against CRC cells is associated with the regulation of the BCL-2 family. The WB results showed that expression of all BCL-2 proteins was attenuated upon treatment of PCD2-Lipo in DLD-1 and CT26 cells, and BCL-XL was the most potently inhibited (FIG. 2E). PS-Lipo may be involved in the regulation of the BCL-2 family, especially BCL-XL. In particular, while free PS was highly cytotoxic to both cancer cells and normal cells, PS-Lipo showed enhanced cytotoxicity against CRC cells while being much less toxic to normal macrophages. These results demonstrate that liposomal encapsulation of free PS confers enhanced antitumor efficacy, particularly toward CRC cells, differently from normal cells.

### 2.3. PCD2-Lipo Antitumor Efficacy is Dependent on the Caspase-9/-3 Axis

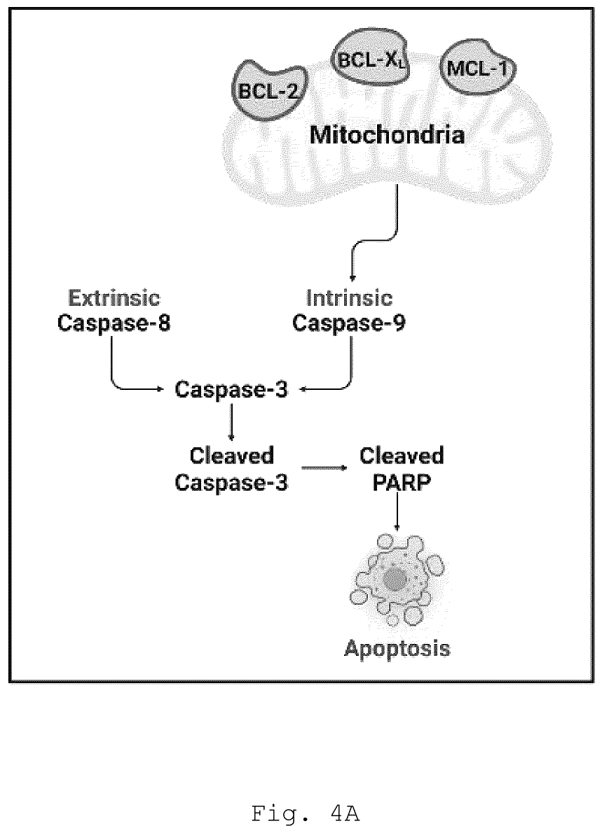

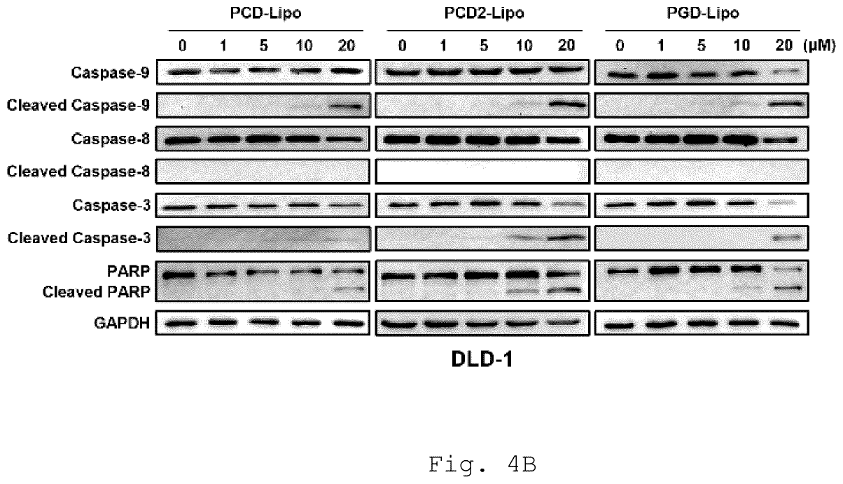

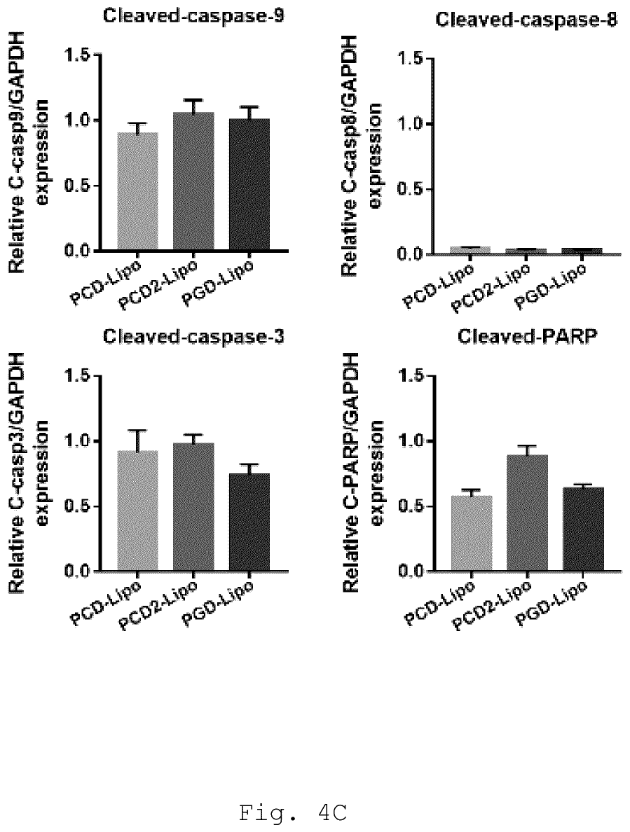

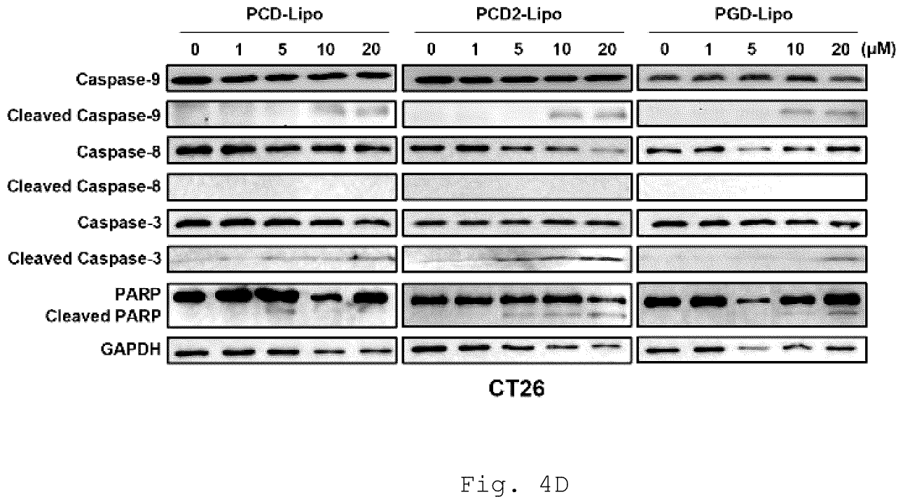

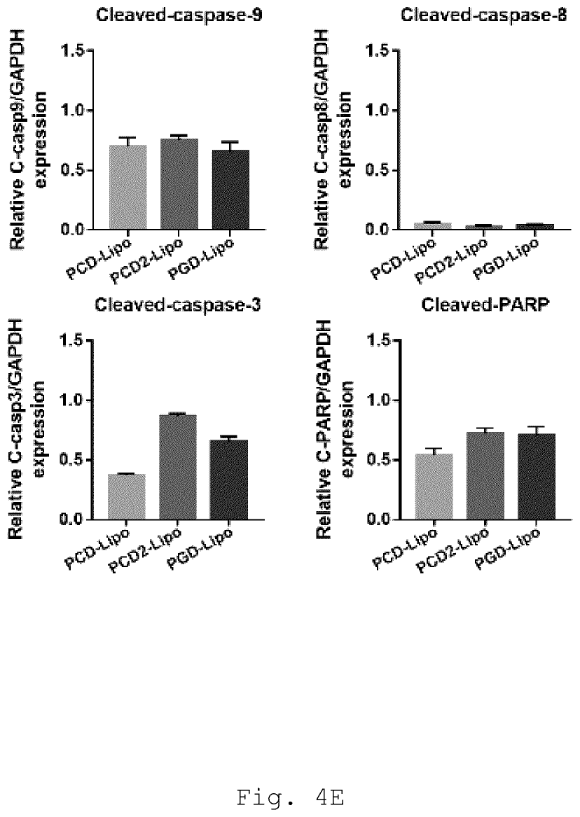

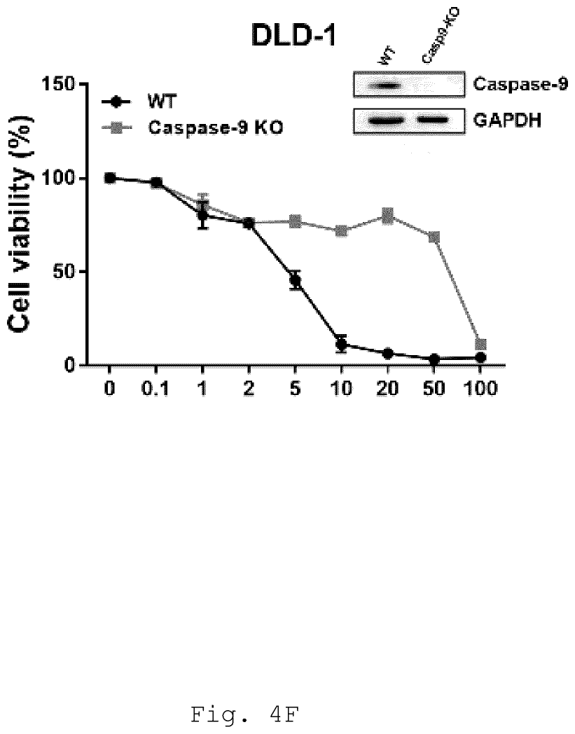

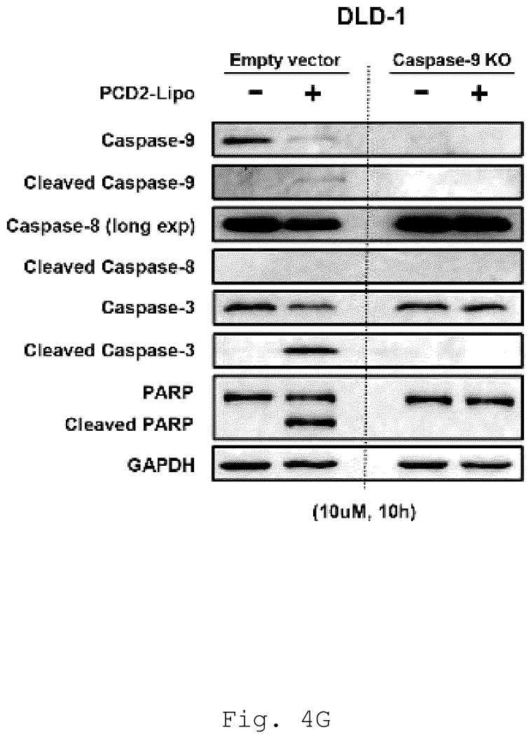

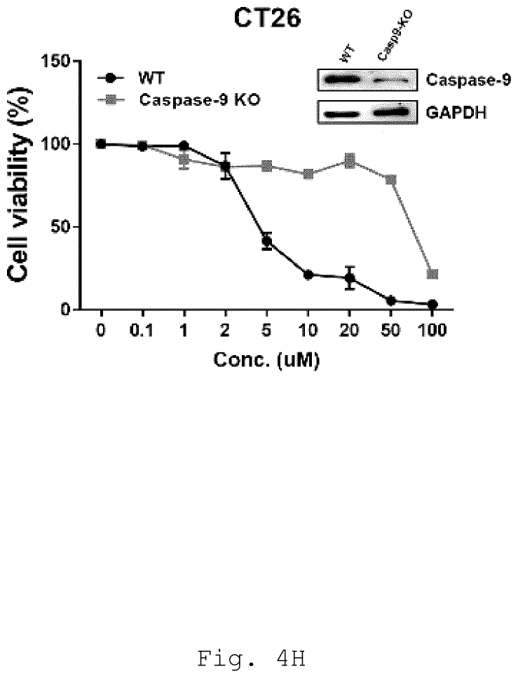

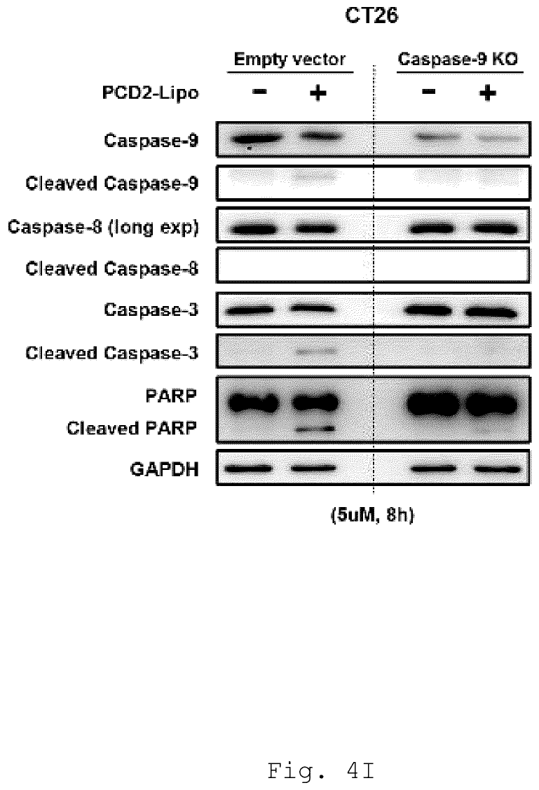

Since PS-Lipo exhibited the highest cytotoxicity against CRC cells (average IC₅₀ value of 22.88 μM) as compared to the other cancer cells (average IC₅₀ value of 52.13 μM), CRC cells were selected for the next study. The cytotoxic effect of PS-Lipo on CRC cells may be further explained by changes in cell death. Platycosides have been reported to exert anticancer activity via the apoptotic signaling pathway. As key mediators of apoptosis, caspase-8, caspase-9, and caspase-3 are located at pivotal junctions in apoptosis pathways (FIG. 4A). The extrinsic pathway is mediated by caspase-8, while the intrinsic pathway may be initiated through caspase-9, and both pathways converge at caspase-3 (executioner), finally resulting in apoptotic cell death. Therefore, a WB analysis was used to investigate the molecular mechanisms of the cytotoxic and apoptotic effects of PS-Lipo. The expression levels of apoptosis-related proteins (caspase-3, caspase-8, caspase-9, and PARP) were detected in PS-Lipo treated DLD-1 and CT26 cells. As shown in FIGS. 4B to 4E, PS-Lipo increased the expression of cleaved caspase-9, cleaved caspase-3 and cleaved PARP in a dose- and time-dependent manner, while cleaved caspase-8 showed no change in expression. These results suggest that PS-Lipo exerted cytotoxicity against CRC cells by inducing caspase-9/-3 dependent apoptosis.

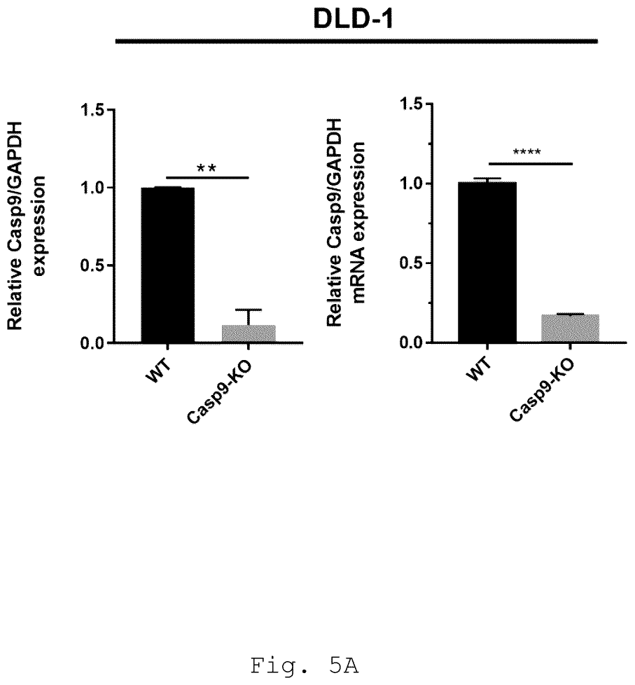

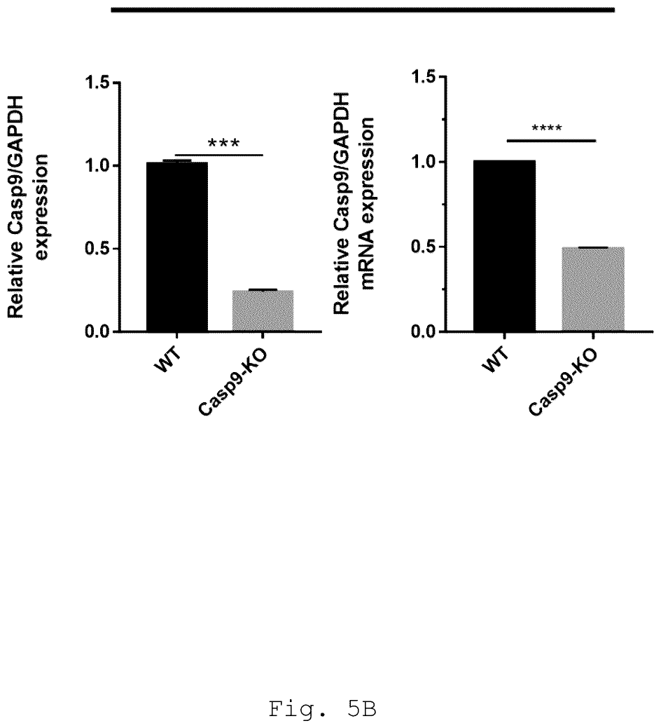

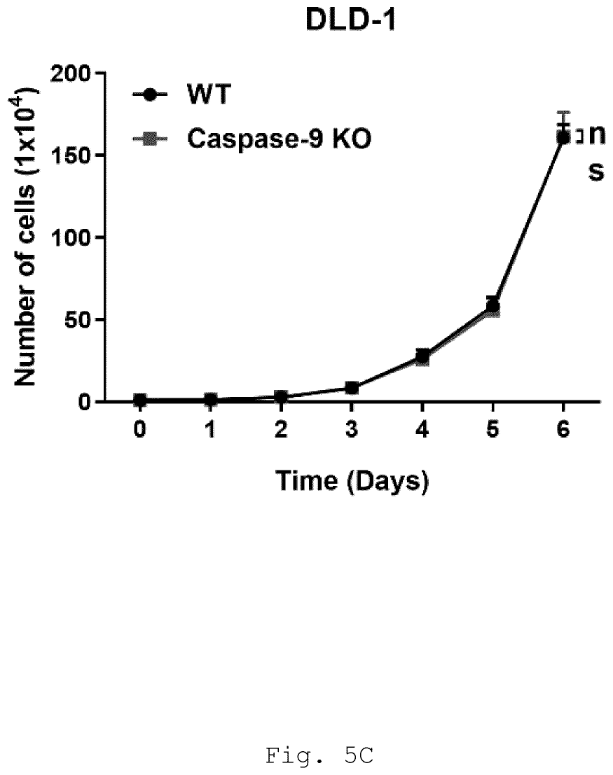

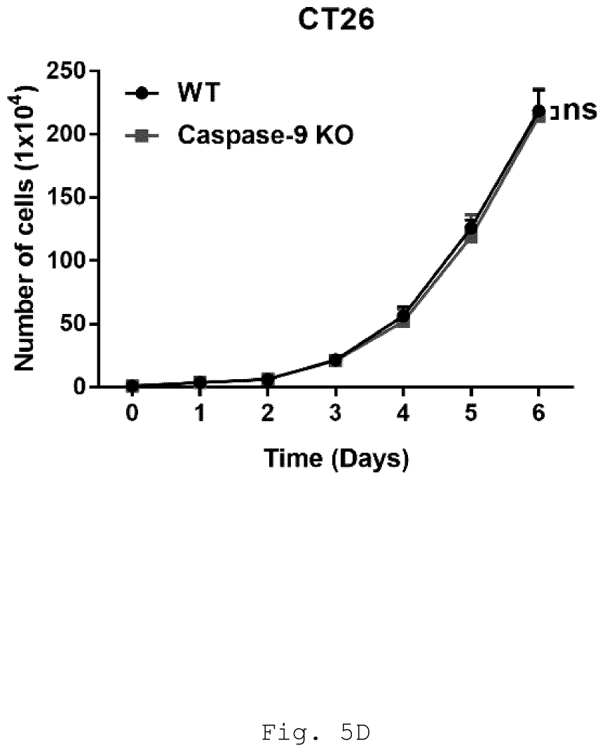

To further evaluate whether PS-Lipo-induced apoptosis is dependent on the caspase-9/-3 axis, caspase-9 knockout (KO) DLD-1 and CT26 cells were generated through CRISPR/Cas9-mediated gene editing (FIGS. 5A and 5B) and the treated with PCD2-Lipo, and cell viability was assessed by performing a WB analysis. Caspase-9 KO DLD-1 and CT26 cells showed normal growth rates similar to wild-type (WT) DLD-1 and CT26 cells (FIGS. 5C and 5D). Since the stability, size, and IC₅₀ of PCD2-Lipo were more favorable than those of other PS-Lipo molecules (FIGS. 1 and 2), PCD2-Lipo was selected in the present study. It was observed that after PCD2-Lipo treatment, the cell viability of WT DLD-1 cells decreased in a dose-dependent manner. However, CRISPR/Cas9 KO of caspase-9 reversed the tumor growth inhibitory effect of PS-Lipo (FIGS. 4F & 4H), indicating that caspase-9 is essentially involved in the growth inhibitory function of PCD2-Lipo.

In many situations, even when a particular caspase is inhibited, apoptosis still occurs by different mechanisms. For example, in the case of the inhibition of the initiator caspase, caspase-9, the executioner caspase-3 may still be activated by caspase-8, which is responsible for the extrinsic pathway. Considering that PCD2-Lipo may not exert cytotoxicity against CRC cells under inhibition of caspase-9, it was attempted to determine whether the failure of PCD2-Lipo-induced antitumor activity is correlated with the inhibition of the caspase-9/-3 axis. Thus, a WB analysis was used to evaluate the expression levels of apoptotic factors under the caspase-9 KO conditions through WT cells. As a result, it was confirmed that the intrinsic apoptotic pathway through the caspase-9/-3 axis was significantly activated in PCD2-Lipo-treated WT DLD-1 and CT26 cells, whereas this activation of the signaling pathway was abolished in caspase-9 KO DLD-1 (FIGS. 4G and 4I). These findings indicate that increased levels of caspase-9, caspase-3, and PARP are associated with the inhibition of tumor growth, but caspase-8 does not play an important role in this process (FIGS. 4G and 4I). In addition, the expression level of the caspase-8/-3 axis was not investigated in both the WT and caspase-9 KO cells. Collectively, these results demonstrated that the caspase-9/-3-mediated intrinsic pathway is particularly responsible for inducing apoptosis in PCD2-Lipo-treated DLD-1 cells.

### 2.4. Tumor-Targeting PCD2-Lipo-ATRAM Inhibits Tumor Growth in CRC Xenografts

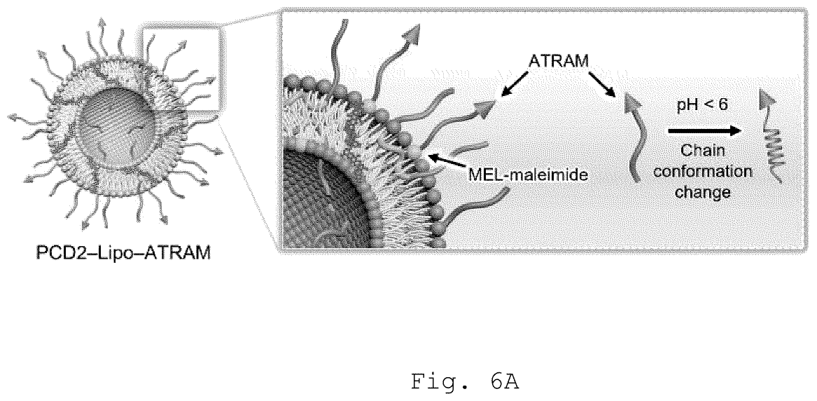

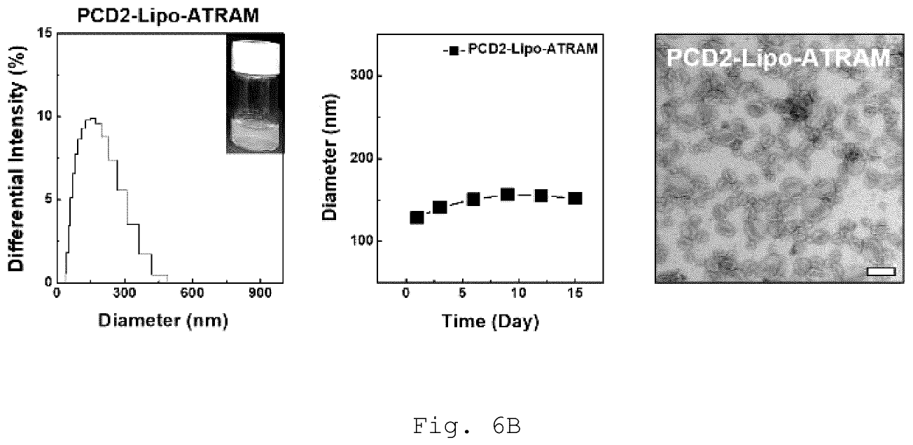

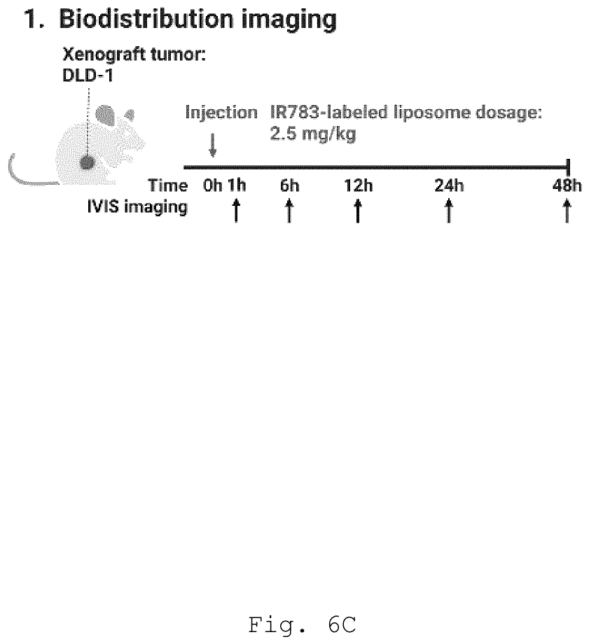

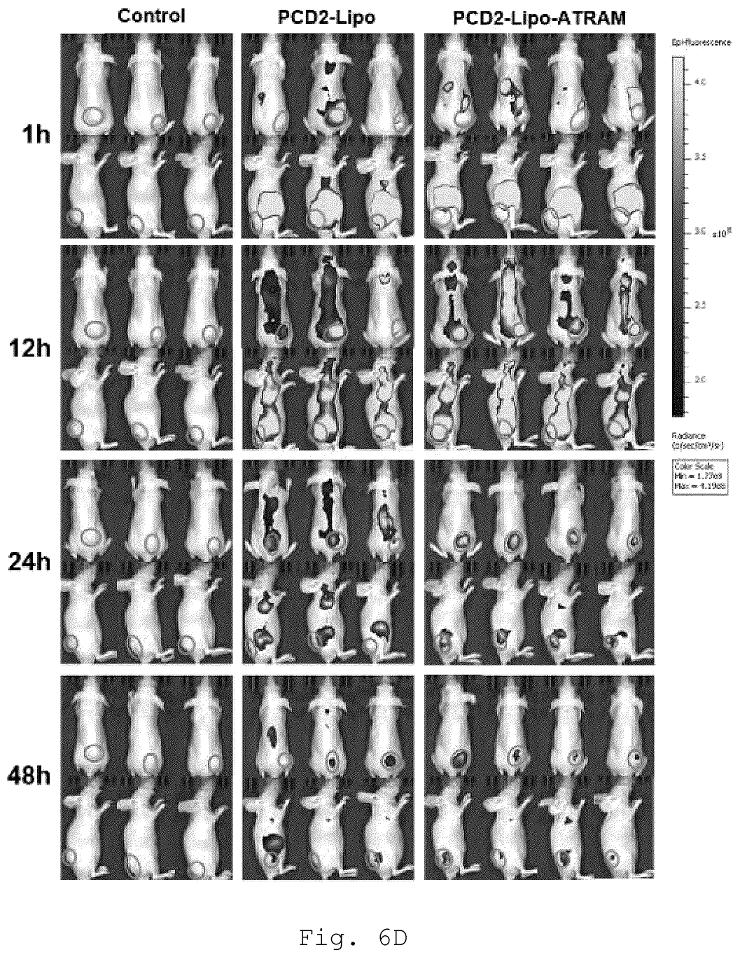

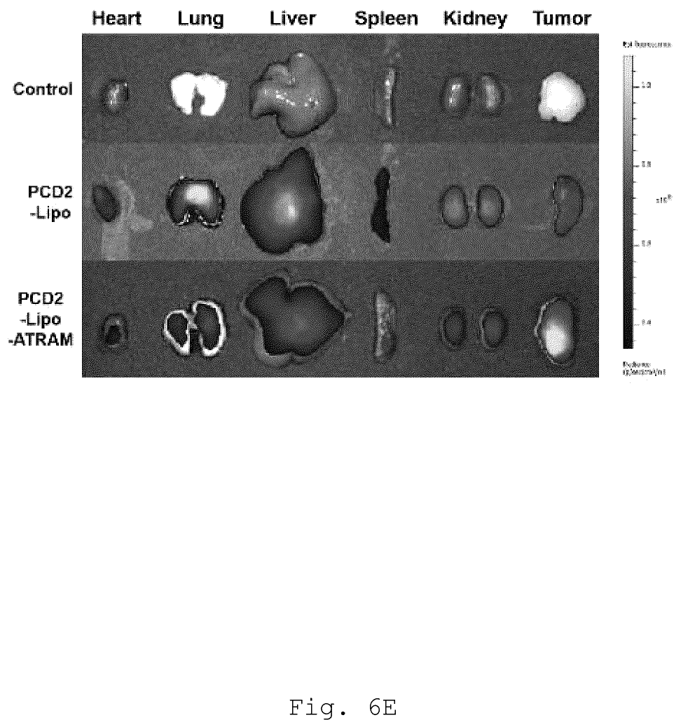

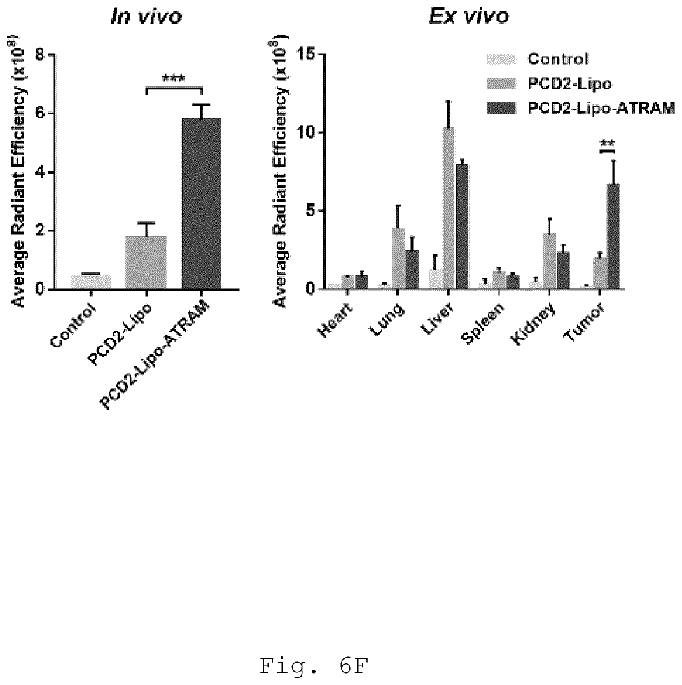

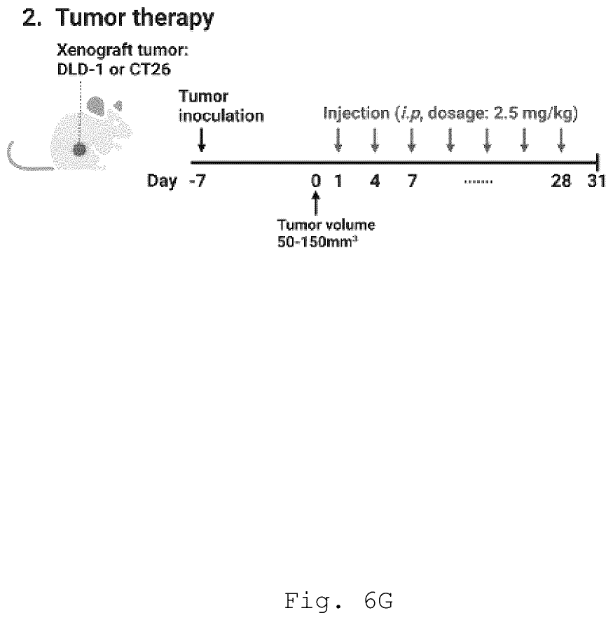

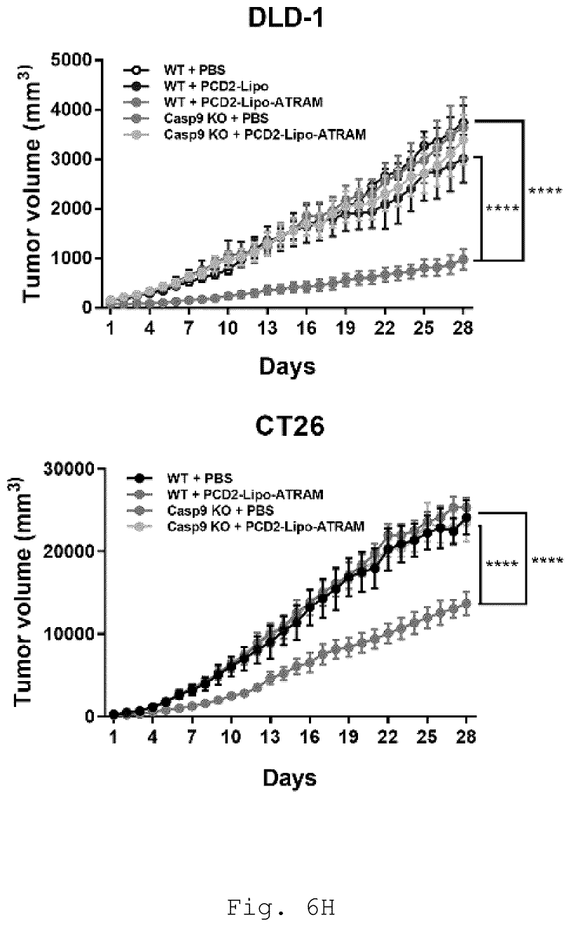

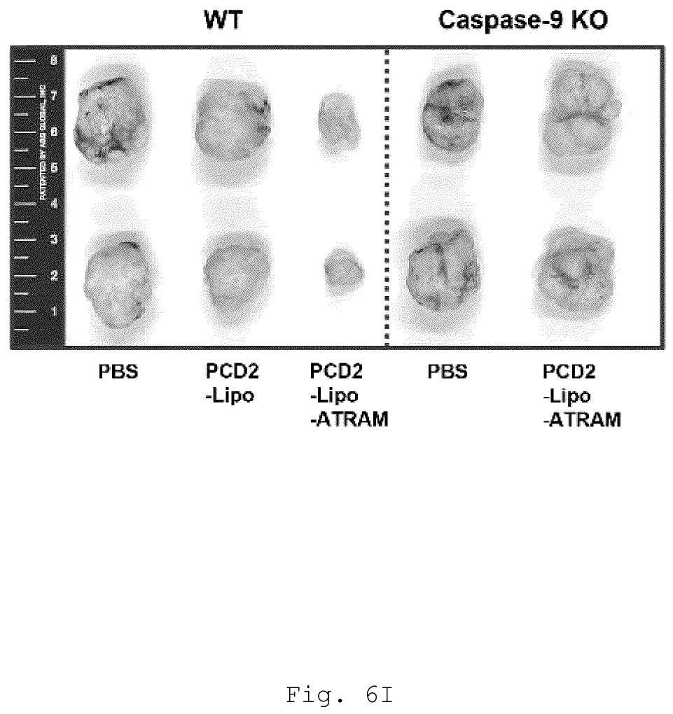

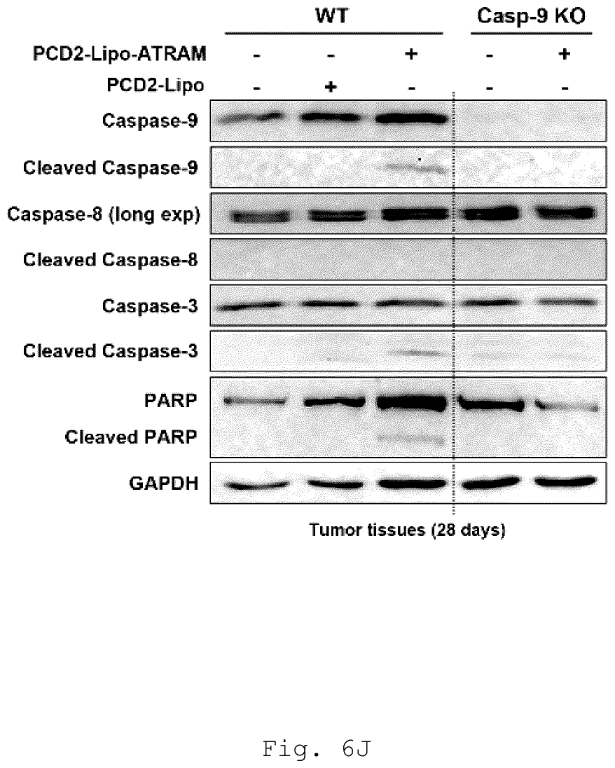

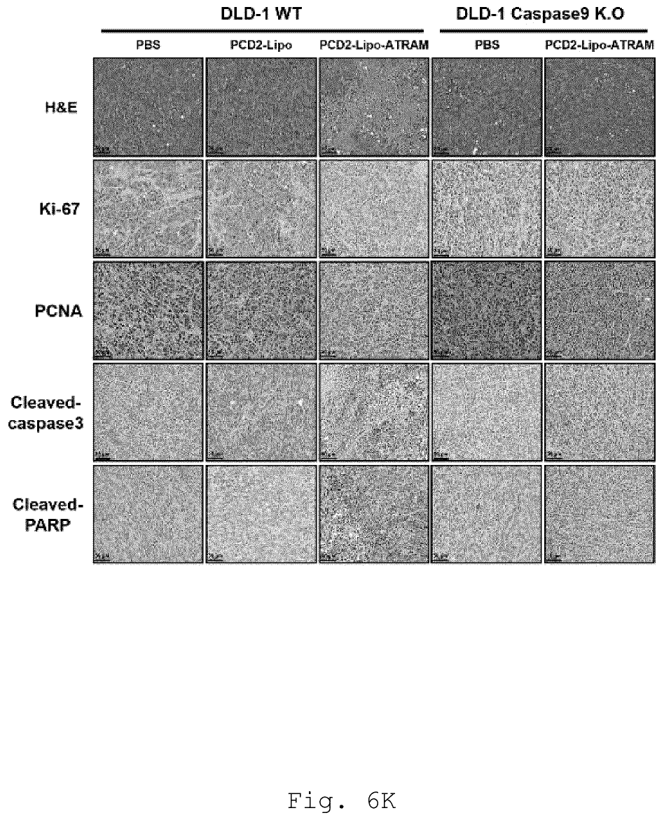

The acidic tumor microenvironment (TME) has been observed to act as a biological barrier during in vivo drug delivery, which blocks the accumulation of nanotherapeutics at tumor sites. To enhance the in vivo tumor-targeting ability, PCD2-Lipo was conjugated with an ATRAM peptide to facilitate the internalization of the liposome specifically into tumor cells in the TME. The ATRAM peptide is known to form a transmembrane helical structure which effectively penetrates into the phospholipid bilayer of the cell membrane under acidic conditions. To deposit the ATRAM peptides on the surface of PCD2-Lipo, a mannosylerythritol lipid (MEL)-maleimide linker was synthesized through the 1-ethyl-3-(-3-dimethyl aminopropyl) carbodiimide (EDC)/N-hydroxysuccinimide coupling reaction. The MEL-maleimide linker may be directly co-assembled with the lipid layer of liposomes. More specifically, a di-alkyl chain of the MEL-maleimide is present in the hydrophobic region of the lipid bilayer, and the hydrophilic maleimide head group is exposed to the aqueous phase. This enables the thiol-terminated ATRAM peptide to be conjugated with the PCD2-Lipo (PCD2-Lipo-ATRAM) via a thiol-maleimide reaction (FIG. 6A). The measured hydrodynamic particle size of PCD2-Lipo-ATRAM was 128.5 nm and the PDI value was 0.353, which were not significantly different from those of PCD2-Lipo, indicating that the conjugation of the ATRAM peptide does not affect the physical characteristics of PCD2-Lipo.

In addition, the vesicular structure of PCD2-Lipo-ATRAM was confirmed through TEM observation (FIG. 6B). To investigate the in vivo tumor-targeting ability of the functionalized PCD2-Lipo-ATRAM, near-infrared (NIR) fluorescence imaging was performed using a DLD-1 xenograft model (FIG. 6C). PCD2-Lipo-ATRAM was labeled with IR-783 (PCD2-Lipo-ATRAM/IR783), an NIR lipid dye, and intraperitoneally injected into DLD-1 tumor-bearing nude mice. Control-Lipo and PCD2-Lipo were used as controls. IVIS Lumina II was used to perform in vivo and ex vivo NIR imaging at different time points after injection. After individual IR783-labeled liposomes were injected, the PCD2-Lipo-ATRAM group showed a much stronger fluorescence signal in the tumor region at all time points (FIGS. 6D to 6F), compared with the control liposomes, indicating that the accumulation of PCD2-Lipo in the tumor region was significantly enhanced by surface functionalization of the liposomes through the ATRAM peptide. PCD2-Lipo without the ATRAM peptide showed almost no fluorescence signal. These results clearly demonstrated the in vivo tumor-targeting effect of PCD2-Lipo-ATRAM.

The antitumor efficacy of PCD2-Lipo-ATRAM was investigated in xenograft models of DLD-1 and CT26 cells. After it was confirmed that the caspase-9/-3 axis plays a crucial role in PCD2-Lipo-induced apoptosis in DLD-1 and CT26 cells in vitro (FIG. 4), DLD-1 WT or caspase-9 KO clones (negative controls) were subcutaneously implanted into the flanks of nude mice (FIG. 6G). After the tumors reached about 50 to 150 mm³, the xenografts were each treated with vehicle, PCD2-Lipo, or PCD2-Lipo-ATRAM, at a dosage of 2.5 mg kg⁻¹ via intraperitoneal injection. As shown in FIGS. 6H and 6I, after a 28-day treatment regimen, PCD2-Lipo-ATRAM exhibited a significant growth inhibitory effect on WT DLD-1 and CT26 tumors, whereas it did not show a significant growth inhibitory effect on caspase-9 KO tumors that exhibited a tumor growth rate comparable to that of WT tumors in vehicle-treated mice. In addition, consistent with the observations in biodistribution studies, PCD2-Lipo without the ATRAM peptide failed to have a significant effect on WT tumor growth, suggesting that surface functionalization of the liposomes by the ATRAM peptide significantly increased the in vivo antitumor efficacy of PCD2-Lipo.

To confirm whether PCD2-Lipo-ATRAM efficacy is dependent on the caspase-9/-3 pathway, WB and immunohistochemistry analyses were performed with control or caspase-9 KO mice-derived tumor tissue. The WB analysis showed that the intrinsic caspase-9/-3 axis induced apoptosis but the extrinsic caspase-8/-3 axis did not correlate with a reduction in tumor growth (FIG. 6J), which was further verified by the detection of caspase-9, caspase-3, and PARP cleavage by H&E staining (FIG. 6K). Collectively, the present invention clearly demonstrated that PCD2-Lipo-ATRAM successfully targeted the tumor site, and thereby exerted a potent antitumor effect on CRC xenografts and that the mechanism of this antitumor effect was dependent on the intrinsic caspase-9/-3 axis.

For example, for the purpose of constituting claims, the claims set forth below should not be construed in any way narrower than their literal language, and thus exemplary embodiments from the specification should not be read as claims. Therefore, it should be understood that the present invention has been described by way of example and not as a limitation on the scope of the claims. Therefore, the presented invention is limited only by the following claims. All publications, published patents, patent applications, books, and journal articles cited in the present application are each incorporated in the present application by reference in their entirety.

1. A pharmaceutical composition for treating cancer, comprising platycodin D2 as a pharmaceutically active ingredient.

2. The pharmaceutical composition of claim 1, wherein the cancer is colorectal cancer, breast cancer, or lung cancer.

3. The pharmaceutical composition of claim 1, wherein the platycodin D2 is encapsulated in a liposome.

4. The pharmaceutical composition of claim 3, wherein the platycodin D2 is fixed to a lipid bilayer of the liposome.

5. The pharmaceutical composition of claim 3, wherein the liposome further includes an acidity-triggered rational membrane (ATRAM) protein.

6. The pharmaceutical composition of claim 5, wherein the ATRAM protein includes an amino acid sequence represented by SEQ ID NO: 1.

7. A liposome comprising platycodin D2.

8. The liposome of claim 7, wherein the platycodin D2 is fixed to a lipid bilayer of the liposome.

9. The liposome of claim 7, further comprising an acidity-triggered rational membrane (ATRAM) protein.

10. The liposome of claim 9, wherein the ATRAM protein includes an amino acid sequence represented by SEQ ID NO: 1.

11. A method of treating cancer, comprising administering a pharmaceutically effective amount of platycodin D2 to a patient in need thereof.

12. The method of claim 11, wherein the cancer is colorectal cancer, breast cancer, or lung cancer.

13. The method of claim 11, wherein the platycodin D2 is encapsulated in a liposome.

14. The method of claim 13, wherein the platycodin D2 is fixed to a lipid bilayer of the liposome.

15. The method of claim 13, wherein the liposome further includes an acidity-triggered rational membrane (ATRAM) protein.

16. The method of claim 15, wherein the ATRAM protein includes an amino acid sequence represented by SEQ ID NO: 1.


SEQUENCE LISTING (9 sequences)

SEQ ID NO: 1 - protein, 34 aa, synthetic construct
GLAGLAGLLGLEGLLGLPLGLLEGLWLGLELEGN

SEQ ID NO: 2 - DNA, 21 bp, synthetic construct
  source 1..21: mol_type = other DNA
catttcatggtggaggtgaag

SEQ ID NO: 3 - DNA, 18 bp, synthetic construct
  source 1..18: mol_type = other DNA
gggaactgcaggtggctg

SEQ ID NO: 4 - DNA, 23 bp, synthetic construct
  source 1..23: mol_type = other DNA
ggtgtgaaccatgagaagtatga

SEQ ID NO: 5 - DNA, 22 bp, synthetic construct
  source 1..22: mol_type = other DNA
gagtccttccacgataccaaag

SEQ ID NO: 6 - DNA, 20 bp, synthetic construct
  source 1..20: mol_type = other DNA
agttcccgggtgctgtctat

SEQ ID NO: 7 - DNA, 20 bp, synthetic construct
  source 1..20: mol_type = other DNA
gccatggtctttctgctcac

SEQ ID NO: 8 - DNA, 20 bp, synthetic construct
  source 1..20: mol_type = other DNA
actccactcacggcaaattc

SEQ ID NO: 9 - DNA, 20 bp, synthetic construct
  source 1..20: mol_type = other DNA
tctccatggtggtgaagaca